(19) United States
(12) Patent Application Publication
(10) Pub. No.: US 2008/0221097 A1
(43) Pub. Date: Sep. 11, 2008

(54) Imidazopyridine Derivatives as Cannabinoid Receptor Ligands

(75) Inventors: Andrew John Eatherton, Essex (GB); Gerard Martin Paul Giblin, Essex (GB); William Leonard Mitchell, Essex (GB); Alan Naylor, Essex (GB); Lee William Page, Essex (GB); Martin Swarbrick, Essex (GB); Jennifer Anne Sweeting, Essex (GB)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(73) Assignee: GLAXO GROUP LIMITED

(21) Appl. No.: 11/997,918

(22) PCT Filed: Aug. 7, 2006
(86) PCT No.: PCT/EP2006/007812

§ 371 Date: Feb. 5, 2008

(30) Foreign Application Priority Data
Aug. 9, 2005 (GB) 0516380.3
Nov. 29, 2005 (GB) 0524324.1

(51) Int. Cl. A61K 31/5377; C07D 471/04; A61P 25/02; A61P 25/04; A61K 31/437

(52) U.S. Cl. 514/234.2; 546/118; 514/303; 544/127

(57) ABSTRACT

The present invention relates to novel imidazopyridine derivatives, pharmaceutical compositions containing these compounds and their use in the treatment of diseases, particularly pain, which diseases are caused directly or indirectly by an increase or decrease in activity of the cannabinoid receptor.

The present invention relates to novel imidazopyridine derivatives, pharmaceutical compositions containing these compounds and their use in the treatment of diseases, particularly pain, which diseases are caused directly or indirectly by an increase or decrease in activity of the cannabinoid receptor.

Cannabinoids are a specific class of psychoactive compounds present in Indian cannabis (Cannabis sativa), including about sixty different molecules, the most representative being cannabinol, cannabidiol and several isomers of tetrahydrocannabinol. Knowledge of the therapeutic activity of cannabis dates back to the ancient dynasties of China, where, 5,000 years ago, cannabis was used for the treatment of asthma, migraine and some gynaecological disorders. These uses later became so established that, around 1850, cannabis extracts were included in the US Pharmacopaeia and remained there until 1947.

Cannabinoids are known to cause different effects on various systems and/or organs, the most important being on the central nervous system and on the cardiovascular system. These effects include alterations in memory and cognition, euphoria, and sedation. Cannabinoids also increase heart rate and vary systemic arterial pressure. Peripheral effects related to bronchial constriction, immunomodulation, and inflammation have also been observed. The capability of cannabinoids to reduce intraocular pressure and to affect respiratory and endocrine systems is also well documented. See e.g. L. E. Hollister, Health Aspects of Cannabis, Pharmacological Reviews, Vol. 38, pp. 1-20, (1986). More recently, it was found that cannabinoids suppress the cellular and humoral immune responses and exhibit antiinflammatory properties. Wirth et al., Antiinflammatory Properties of Cannabichrome, Life Science, Vol. 26, pp. 1991-1995, (1980).

In spite of the foregoing benefits, the therapeutic use of cannabis is controversial, both due to its relevant psychoactive effects (causing dependence and addiction), and due to manifold side effects that have not yet been completely clarified. Although work in this field has been ongoing since the 1940's, evidence indicating that the peripheral effects of cannabinoids are directly mediated, and not secondary to a CNS effect, has been limited by the lack of receptor characterization, the lack of information concerning an endogenous cannabinoid ligand and, until recently, the lack of receptor subtype selective compounds.

The first cannabinoid receptor was found to be mainly located in the brain, in neural cell lines, and, only to a lesser extent, at the peripheral level. In view of its location, it was called the central receptor (“CB1”). See Matsuda et al., “Structure of a Cannabinoid Receptor and Functional Expression of the Cloned cDNA,” Nature, Vol. 346, pp. 561-564 (1990). The second cannabinoid receptor (“CB2”) was identified in the spleen, and was assumed to modulate the non psychoactive effects of the cannabinoids. See Munro et el., “Molecular Characterization of a Peripheral Receptor for Cannabinoids,” Nature, Vol. 365, pp. 61-65 (1993).

The foregoing indications and the preferential localization of the CB2 receptor in the immune system confirms a specific role of CB2 in modulating the immune and antiinflammatory response to stimuli of different sources.

The total size of the patient population suffering from pain is vast (almost 300 million), dominated by those suffering from back pain, osteo-arthritic pain and post-operative pain. Neuropathic pain (associated with neuronal lesions such as those induced by diabetes, HIV, herpes infection, or stroke) occurs with lower, but still substantial prevalence, as does cancer pain.

The pathogenic mechanisms that give rise to pain symptoms can be grouped into two main categories:

those that are components of inflammatory tissue responses (Inflammatory Pain):

those that result from a neuronal lesion of some form (Neuropathic Pain).

Chronic inflammatory pain consists predominantly of osteoarthritis, chronic low back pain and rheumatoid arthritis. The pain results from acute and on-going injury and/or inflammation. There may be both spontaneous and provoked pain.

There is an underlying pathological hypersensitivity as a result of physiological hyperexcitability and the release of inflammatory mediators which further potentiate this hyperexcitability. CB2 receptors are expressed on inflammatory cells (T cells, B cells, macrophages, mast cells) and mediate immune suppression through inhibition of cellular interaction/inflammatory mediator release. CB2 receptors may also be expressed on sensory nerve terminals and therefore directly inhibit hyperalgesia.

More recently, data suggests a role for CB2 receptor activation in the CNS. Until recently the CB2 receptor was thought to be restricted to the periphery, however emerging data suggests inflammatory pain-mediated induction of CB2 receptor expression in rat spinal cord which coincides with the appearance of activated microglia (Zhang et. al., 2003). Furthermore CB2 receptor agonists have been shown to reduce mechanically evoked responses and wind-up of wide dynamic range neurones in spinal cord dorsal horn in animal models of inflammatory pain (Zhang et. al., 2003, Eur J. Neurosci. 17: 2750-2754, Nackley et. al., 2004, J. Neurophys. 92: 3562-3574, Elmes et. al., 2004, Eur. J. Neurosci. 20: 2311-2320.)

The role of CB2 in immunomodulation, inflammation, osteoporosis, cardiovascular, renal and other disease conditions is now being examined.

Based on the foregoing, there is a need for compounds which have activity against the CB2 receptor. Thus, CB2 modulators are believed to offer a unique approach toward the pharmacotherapy of immune disorders, inflammation, osteoporosis, renal ischemia and other pathophysiological conditions.

WO 04/018433, WO 04/018434, WO04/029027 and WO04/029026 (all in the name of Glaxo Group Limited) describe pyrimidine and pyridine derivatives useful n the treatment of diseases which are caused directly or indirectly by an increase or decrease in activity of the cannabinoid receptor.

The present invention provides novel imidazopyridine derivatives of formula (I) and pharmaceutically acceptable derivatives thereof, pharmaceutical compositions containing these compounds or derivatives, and their use as CB2 receptor modulators, which are useful in the treatment of a variety of disorders.

The present invention further comprises a method for treating disease mediated by CB2 receptors in an animal, including humans, which comprises administering to an animal in need thereof an effective, non toxic, amount of a compound of formula (I) or a pharmaceutically acceptable derivative thereof.

In light of the fact that cannabinoids act on receptors capable of modulating different functional effects, and in view of the low homology between CB2 and CB1, a class of drugs selective for the specific receptor sub-type is desirable. The natural or synthetic cannabinoids currently available do not fulfil this function because they are active on both receptors.

In one embodiment the present invention includes compounds which are capable of selectively modulating the receptors for cannabinoids and therefore the pathologies associated with such receptors.

The invention provides compounds of formula (I):

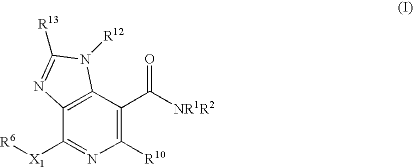

wherein:

X₁ is NR⁴ or O;

R¹ is selected from hydrogen, C₁₋₆alkyl, C₃₋₆cycloalkyl and halosubstitutedC₁₋₆alkyl;

R² is hydrogen or (CH₂)ₘR³ where m is 0 or 1;

or R¹ and R² together with N to which they are attached form an optionally substituted 4- to 8-membered non-aromatic heterocyclyl ring;

R³ is a 4- to 8-membered non-aromatic heterocyclyl group, a C₃₋₈ cycloalkyl group, a straight or branched C₁₋₁₀alkyl, a C₂₋₁₀ alkenyl, a C₃₋₈cycloalkenyl, a C₂₋₁₀alkynyl, a C₃₋₈cycloalkynyl or phenyl group, any of which can be unsubstituted or substituted, or R⁵;

R⁴ is selected from hydrogen, C₁₋₆ alkyl, C₃₋₆ cycloalkyl, halosubstitutedC₁₋₆ alkyl, COCH₃and SO₂Me;

R⁵ is

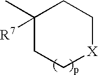

wherein p is 0, 1 or 2, and X is CH₂O, S, or SO₂;

R⁶ is unsubstituted or substituted phenyl, unsubstituted or substituted C₃₋₆cycloalkyl or an unsubstituted or substituted 4- to 8-membered non-aromatic heterocyclyl ring;

R⁷ is OH, C₁₋₆alkoxy, NR^{8a}R^{8b}, NHCOR⁹, NHSO₂R⁹ or SOqR⁹;

R^{8a} is H or C₁₋₆alkyl;

R^{8b} is H or C₁₋₆alkyl;

R⁹ is C₁₋₆alkyl;

R¹⁰ is hydrogen, substituted or unsubstituted (C₁₋₆)alkyl or chloro;

R¹² is hydrogen or C₁₋₆alkyl;

R¹³ is hydrogen or C₁₋₆alkyl;

q is 0, 1 or 2;

and pharmaceutically acceptable derivatives thereof.

In one embodiment R¹ is hydrogen.

In one embodiment R² is (CH₂)ₘR³ where m is 0 or 1.

In one embodiment X₁ is NR⁴.

In one embodiment X₁ is O.

When R³ or R⁶ are independently selected from a non-aromatic heterocyclyl group, the ring may contain 1, 2, 3, or 4 hetero atoms. In one embodiment the hetero atoms are selected from oxygen, nitrogen or sulphur. Examples of 4-membered groups are 2- or 3-azetidinyl, oxetanyl, thioxetanyl, thioxetanyl-s-oxide and thioxetanyl-s,s-dioxide. Examples of 5-membered heterocyclyl groups in this instance include dioxolanyl, pyrrolidinyl, tetrahydrofuranyl, tetrahydrothiophenyl, tetrahydrothiophenyl-s,s-dioxide and tetrahydrothiophenyl-s-oxide. Examples of 6-membered heterocyclyl groups are morpholinyl, piperidinyl, piperazinyl, tetrahydropyranyl, tetrahydrothiopyranyl, tetrahydrothiopyranyl-s,s-dioxide, thiomorpholinyl, thiomorpholinyl-s,s-dioxide, tetrahydropyridinyl, dioxanyl, tetrahydrothiopyran-1,1-dioxide and tetrahydrothiopyran-1-oxide. Examples of a 7-membered heterocyclyl ring are azapine or oxapine. Examples of 8-membered groups are azacyclooctanyl, azaoxacyclooctanyl or azathiacyclooctanyl, oxacylcooctanyl, thiacyclooctanyl and azathiacyclooctanyl-s-oxide, azathiacyclooctanyl-s,s-dioxide, thiacyclooctanyl-s,s-dioxide, and thiacyclooctanyl-s-oxide.

In one embodiment R³ is an unsubstituted or substituted C₁₋₆ alkyl group.

In one embodiment R⁴ is C₁₋₆ alkyl or hydrogen, for example methyl or hydrogen.

In one embodiment R⁴ is hydrogen.

When R¹ and R² taken together with the N to which they are attached form an optionally substituted non-aromatic heterocyclyl ring the ring may optionally contain 1, 2, 3 or 4 further hetero atoms. The ring may be saturated or unsaturated. In one embodiment the further hetero atoms are selected from oxygen, nitrogen or sulphur. An example of a 4-membered heterocyclyl ring is azetidinyl. Examples of a 5-membered heterocyclyl ring are pyrrolidinyl and pyrazolidinyl. Examples of 6-membered heterocyclyl rings are morpholinyl, piperazinyl, piperidinyl, tetrahydropyridinyl, thiomorpholine-s,s-dioxide, thiomorpholinyl and thiomorpholinyl-s-oxide. Examples of a 7-membered heterocyclyl ring are azapine or oxapine. Examples of 8-membered heterocyclyl rings are azacyclooctanyl, azaoxacyclooctanyl or azathiacyclooctanyl.

In one embodiment, R¹ and R² together with the nitrogen to which they are attached form a morpholinyl, pyrrolidinyl or piperidinyl ring. In another embodiment, R¹ and R² together with the nitrogen to which they are attached form a morpholinyl ring.

In one embodiment R⁶ is an unsubstituted or substituted phenyl.

In one embodiment R⁷ is OH.

In one embodiment R¹⁰ is hydrogen.

In one embodiment R¹² is methyl or hydrogen. In another embodiment R¹² is methyl.

In one embodiment R¹³ is methyl or hydrogen. In another embodiment R¹³ is hydrogen.

When R⁶ is substituted, it may be substituted by 1, 2 or 3 substituents, the substituent or substituents may be selected from: C₁₋₆ alkyl, halosubstitutedC₁₋₆ alkyl e.g. trifluoromethyl, C₁₋₆ alkoxy, a hydroxy group, a cyano group, halo, a C₁₋₅₆alkyl sulfonyl group, —CONH₂,—NHCOCH₃, —COOH, halosubstituted C₁₋₆ alkoxy e.g. trifluoromethyloxy and SO₂NR^{8a}R^{8b}wherein R^{8a} and R^{8b} are as defined above.

In one embodiment R⁶ is substituted by 1 or 2 substituents.

In one embodiment R⁶ is substituted by substitutents selected from halo, cyano, methyl, trifluoromethyl, methoxy and trifluoromethoxy.

In one embodiment R⁶ is substituted by halo, for example chloro. In another embodiment R⁶ is 3-chlorophenyl.

When R¹ and R² together with N to which they are attached form a 4- to 8-membered non-aromatic heterocyclyl ring which is substituted, or when R³ is substituted, the substituent or substituents may be selected from: C₁₋₆ alkyl, C₁₋₆ alkoxy, a hydroxy group, halosubstituted C₁₋₆alkyl e.g. trifluoromethyl, halosubstituted C₁₋₆alkoxy e.g. trifluoromethyloxy, a cyano group, halo or a sulfonyl group, methylsulfonyl, NR^{8a} R^{8b}, CONH₂, NHCOCH3 (═O), COOH, CONHCH₃CON(CH₃)₂ and NHSO₂CH₃ wherein R^{8a} and R^{8b} are as described above.

When R¹ and R² together with N to which they are attached form a 4- to 8-membered non-aromatic heterocyclyl ring which is substituted, or when R³ is substituted there can be 1, 2 or 3 substituents.

When R¹⁰ is substituted, the substituents may be selected from halogen.

In one embodiment the invention is compounds of formula (Ia);

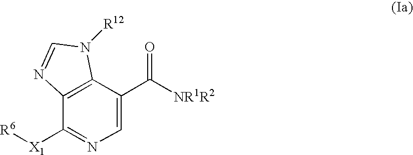

wherein

X₁ is NR⁴;

R¹ is hydrogen;

R² is (CH₂)ₘR³where m is 0 or 1;

or R¹ and R² together with N to which they are attached form a morpholinyl, pyrrolidinyl, or piperidinyl ring any of which may be unsubstituted or substituted;

R³ is an unsubstituted or substituted straight or branched C₁₋₆alkyl;

R⁴ is hydrogen or methyl,

R⁶ is unsubstituted or substituted phenyl;

R¹² is hydrogen or methyl;

and pharmaceutically acceptable derivatives thereof.

In certain embodiments compounds of formula (I) show selectivity for CB2 over CB1.

In one embodiment compounds of formula (I) have an EC50 value at the cloned human cannabinoid CB2 receptor of at least 50 times the EC50 values at the cloned human cannabinoid CB1 receptor and/or have less than 10% efficacy at the CB1 receptor.

In one embodiment compounds of formula (I) have an EMR value at the cloned human cannabinoid CB2 receptor of at least 5 times the EMR value at the cloned human cannabinoid CB1 receptor. In another embodiment compounds of formula (I) have an EMR value at the cloned human cannabinoid CB2 receptor of at least 10 times the EMR value at the cloned human cannabinoid CB1 receptor. EMR is the equieffective molar ratio and values may be calculated from the equation set out hereinbelow.

Compounds of formula (I) may be more potent and/or more soluble and/or more bioavailable and/or produce a more linear increase in exposure when the compounds are orally administered to a mammal than earlier published compounds which are agonists of CB2.

The invention is described using the following definitions unless otherwise indicated.

The term “pharmaceutically acceptable derivative” means any pharmaceutically acceptable salt, ester, salt of such ester or solvate (including solvates of salts, esters, or salts of esters) of the compounds of formula (I), or any other compound which upon administration to the recipient is capable of providing (directly or indirectly) a compound of formula (I) or an active metabolite or residue thereof. In one embodiment the pharmaceutically acceptable derivative is a salt or solvate of compound of formula (I).

It will be appreciated by those skilled in the art that compounds of formula (I) may be modified to provide pharmaceutically acceptable derivatives thereof at any of the functional groups in the compounds, and that the compounds of formula (I) may be derivatised at more than one position.

It will be appreciated that, for pharmaceutical use, the salts, esters, salts of esters and solvates referred to above will be physiologically acceptable salts, esters, salts of esters and solvates but other salts, esters, salts of esters and solvates may find use, for example in the preparation of compounds of formula (I) and the physiological acceptable salts, esters, salts of esters and solvates thereof. Pharmaceutically acceptable salts include those described by Berge, Bighley and Monkhouse, J. Pharm. Sci., 1977, 66, 1-19. The term “pharmaceutically acceptable salts” includes salts prepared from pharmaceutically acceptable non-toxic bases including inorganic bases and organic bases. Salts derived from inorganic bases include aluminum, ammonium, calcium, copper, ferric, ferrous, lithium, magnesium, manganic salts, manganous, potassium, sodium, zinc, and the like. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, trishydroxylmethyl amino methane, tripropyl amine, tromethamine, and the like. When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric. pamoic, pantothenic, phosphoric, succinic, sulfuric, tartaric, p-toluenesulfonic acid, and the like.

Examples of pharmaceutically acceptable salts include the ammonium, calcium, magnesium, potassium, and sodium salts, and those formed from maleic, fumaric, benzoic, ascorbic, pamoic, succinic, hydrochloric, sulfuric, bismethylenesalicylic, methanesulfonic, ethanedisulfonic, propionic, tartaric, salicylic, citric, gluconic, aspartic, stearic, palmitic, itaconic, glycolic, p-aminobenzoic, glutamic, benzenesulfonic, cyclohexylsulfamic, phosphoric and nitric acids.

The terms ‘halogen or halo’ are used to represent fluorine, chlorine, bromine or iodine.

The term ‘alkyl’ as a group or part of a group means a straight or branched chain alkyl group or combinations thereof, for example a methyl, ethyl, n-propyl, i-propyl, n-butyl, s-butyl, t-butyl, i-butyl, pentyl, hexyl, 1,1-dimethylethyl, heptyl, octyl, nonyl, decyl or combinations thereof.

The term ‘alkoxy’ as a group or as part of a group means a straight, branched or cyclic chain alkyl group having an oxygen atom attached to the chain, for example a methoxy, ethoxy, n-propoxy, i-propoxy, n-butoxy, s-butoxy, t-butoxy group, i-butoxy, pentoxy, hexyloxy group, cyclopentoxy or cyclohexyloxy group.

The term ‘cycloalkyl’ means a closed saturated ring, for example cyclopropyl, cyclobutyl, cyclopentyl, cyclohexyl or cycloheptyl, or cyclooctyl.

The term ‘alkenyl’ as a group or part of a group means a straight or branched chain carbon chain or combinations thereof containing 1 or more double bonds, for example butenyl, pentenyl, hexenyl or heptenyl, or octenyl.

The term ‘cycloalkenyl’ means a closed non-aromatic carbon ring containing 1 or more double bonds, for example cyclobutenyl, cyclopentenyl, cyclohexenyl or cycloheptenyl, or cyclooctenyl.

The term ‘alkynyl’ as a group or part of a group means a straight or branched chain carbon chain or combinations thereof containing 1 or more triple carbon bonds for example ethynyl, propynyl, butynyl, pentynyl, hexynyl or combinations thereof.

The term ‘cycloalkynyl’ means a closed non-aromatic carbon ring containing 1 or more triple carbon bonds for example cyclopropynyl, cyclobutynyl, cyclopentynyl, cyclohexynyl or combinations thereof.

The term ‘aryl’ means a 5- or 6-membered aromatic ring, for example phenyl, or a 7- to 12-membered bicyclic ring system where at least one of the rings is aromatic, for example naphthyl.

The present invention also provides processes for the preparation of compounds of the invention and intermediates (II), (III), (IV), (V), (VI) and (VII) used therein.

Compounds of formula (I) can be prepared as set out in scheme 1:

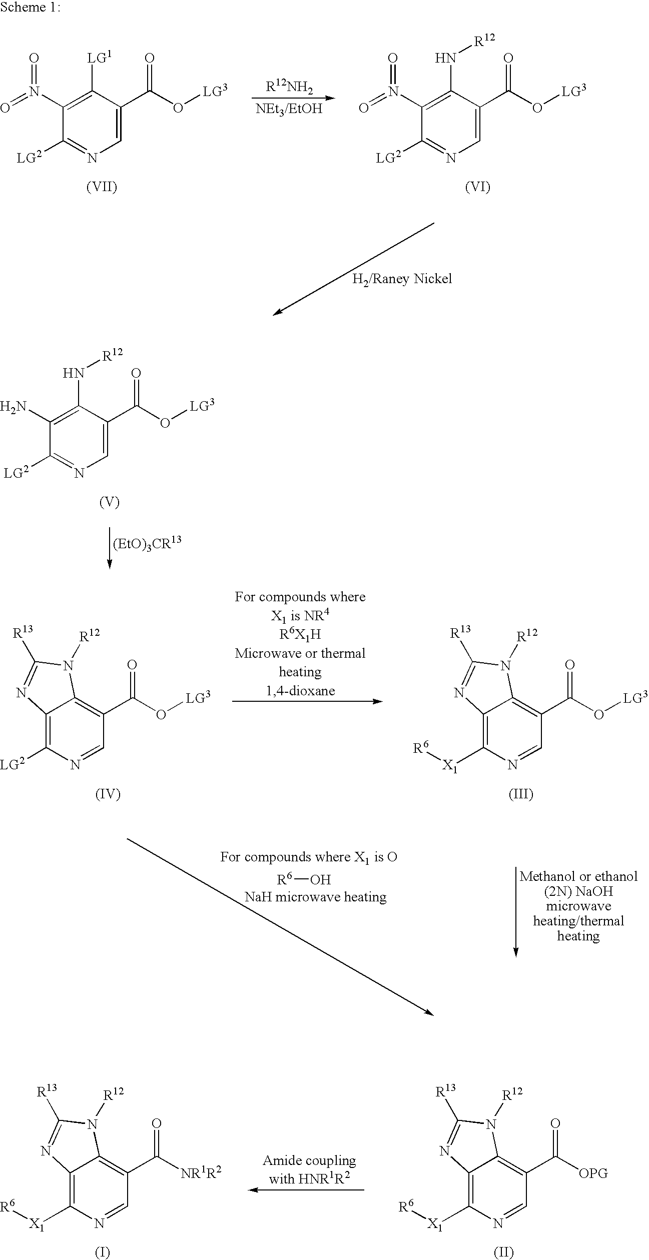

wherein LG¹ and LG² are leaving groups for example halo, eg chlorine, LG³ is a leaving group for example C₁₋₆alkyl e.g methyl or ethyl, PG is hydrogen or an alkaline metal ion eg Na⁺ and X₁, R¹, R², R⁶, R¹² and R¹³ are as defined for compounds of formula (I).

It is to be understood that the present invention encompasses all isomers of compounds of formula (I) and their pharmaceutically acceptable derivatives, including all geometric, tautomeric and optical forms, and mixtures thereof (e.g. racemic mixtures). Where additional chiral centres are present in compounds of formula (I), the present invention includes within its scope all possible diasteroismers, including mixtures thereof. The different isomeric forms may be separated or resolved one from the other by conventional methods, or any given isomer may be obtained by conventional synthetic methods or by stereospecific or asymmetric syntheses.

The subject invention also includes isotopically-labeled compounds, which are identical to those recited in formula (I) and following, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine, iodine, and chlorine, such as ³H, ¹¹C, ¹⁴C, ¹⁸F, ¹²³I and ¹²⁵I.

Compounds of the present invention and pharmaceutically acceptable salts of said compounds that contain the aforementioned isotopes and/or other isotopes of other atoms are within the scope of the present invention. Isotopically-labeled compounds of the present invention, for example those into which radioactive isotopes such as ³H, ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. ¹¹C and ⁸F isotopes are particularly useful in PET (positron emission tomography), and ¹²⁵I isotopes are particularly useful in SPECT (single photon emission computerized tomography), all useful in brain imaging. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of formula (I) and following of this invention can generally be prepared by carrying out the procedures disclosed in the Schemes and/or in the Examples below, by substituting a readily available isotopically labeled reagent for a non-isotopically labeled reagent.

Compounds of formula (I) and their pharmaceutically acceptable derivatives may be prepared in crystalline or non-crystalline form, and, if crystalline, may optionally be solvated. References to solvates herein include hydrates. This invention includes within its scope stoichiometric solvates (including hydrates) as well as compounds containing variable amounts of water and/or solvent.

In view of their ability to bind to the CB2 receptor, it is believed that compounds of the invention will be useful in the treatment of the disorders that follow. Thus, compounds of formula (I) and their pharmaceutically acceptable derivatives may be useful as analgesics. For example they may be useful in the treatment of chronic inflammatory pain (e.g. pain associated with rheumatoid arthritis, osteoarthritis, rheumatoid spondylitis, gouty arthritis and juvenile arthritis) including the property of disease modification and joint structure preservation; musculoskeletal pain; lower back and neck pain; sprains and strains; neuropathic pain; sympathetically maintained pain; myositis; pain associated with cancer and fibromyalgia; pain associated with migraine; pain associated with influenza or other viral infections, such as the common cold; rheumatic fever; pain associated with functional bowel disorders such as non-ulcer dyspepsia, non-cardiac chest pain and irritable bowel syndrome; pain associated with myocardial ischemia; post operative pain; headache; toothache; and dysmenorrhea.

Compounds of the invention may also have disease modification or joint structure preservation properties in multiple sclerosis, rheumatoid arthritis, osteo-arthritis, rheumatoid spondylitis, gouty arthritis and juvenile arthritis.

Compounds of the invention may be particularly useful in the treatment of neuropathic pain. Neuropathic pain syndromes can develop following neuronal injury and the resulting pain may persist for months or years, even after the original injury has healed. Neuronal injury may occur in the peripheral nerves, dorsal roots, spinal cord or certain regions in the brain. Neuropathic pain syndromes are traditionally classified according to the disease or event that precipitated them. Neuropathic pain syndromes include: diabetic neuropathy; sciatica; non-specific lower back pain; multiple sclerosis pain; fibromyalgia; HIV-related neuropathy; post-herpetic neuralgia; trigeminal neuralgia; and pain resulting from physical trauma, amputation, cancer, toxins or chronic inflammatory conditions. These conditions are difficult to treat and although several drugs are known to have limited efficacy, complete pain control is rarely achieved. The symptoms of neuropathic pain are incredibly heterogeneous and are often described as spontaneous shooting and lancinating pain, or ongoing, burning pain. In addition, there is pain associated with normally non-painful sensations such as “pins and needles” (paraesthesias and dysesthesias), increased sensitivity to touch (hyperesthesia), painful sensation following innocuous stimulation (dynamic, static or thermal allodynia), increased sensitivity to noxious stimuli (thermal, cold, mechanical hyperalgesia), continuing pain sensation after removal of the stimulation (hyperpathia) or an absence of or deficit in selective sensory pathways (hypoalgesia).

Compounds of formula (I) and their pharmaceutically acceptable derivatives may also be useful in the treatment of fever.

Compounds of formula (I) and their pharmaceutically acceptable derivatives may also be useful in the treatment of inflammation, for example in the treatment of skin conditions (e.g. sunburn, burns, eczema, dermatitis, psoriasis); ophthalmic diseases such as glaucoma, retinitis, retinopathies, uveitis and of acute injury to the eye tissue (e.g. conjunctivitis); lung disorders (e.g. asthma, bronchitis, emphysema, allergic rhinitis, respiratory distress syndrome, pigeon fancier's disease, farmer's lung, chronic obstructive pulmonary disease, (COPD); gastrointestinal tract disorders (e.g. aphthous ulcer, Crohn's disease, atopic gastritis, gastritis varialoforme, ulcerative colitis, coeliac disease, regional ileitis, irritable bowel syndrome, inflammatory bowel disease, gastroesophageal reflux disease); organ transplantation; other conditions with an inflammatory component such as vascular disease, migraine, periarteritis nodosa, thyroiditis, aplastic anaemia, Hodgkin's disease, sclerodoma, myaesthenia gravis, multiple sclerosis, sorcoidosis, nephrotic syndrome, Bechet's syndrome, polymyositis, gingivitis, myocardial ischemia, pyrexia, systemic lupus erythematosus, tendinitis, bursitis, and Sjogren's syndrome.

Compounds of formula (I) and their pharmaceutically acceptable derivatives may also be useful in the treatment of bladder hyperrelexia following bladder inflammation.

Compounds of formula (I) and their pharmaceutically acceptable derivatives may also be useful in the treatment of immunological diseases such as autoimmune diseases, immunological deficiency diseases or organ transplantation. The compounds of formula (I) and their pharmaceutically acceptable derivatives may also be effective in increasing the latency of HIV infection.

Compounds of formula (I) and their pharmaceutically acceptable derivatives may also be useful in the treatment of diseases of abnormal platelet function (e.g. occlusive vascular diseases).

Compounds of formula (I) and their pharmaceutically acceptable derivatives may also be useful in the treatment of neuritis, heart burn, dysphagia, pelvic hypersensitivity, urinary incontinence, cystitis or pruritis.

Compounds of formula (I) and their pharmaceutically acceptable derivatives may also have diuretic action.

Compounds of formula (I) and their pharmaceutically acceptable derivatives may also be useful in the treatment of impotence or erectile dysfunction.

Compounds of formula (I) and their pharmaceutically acceptable derivatives may also be useful for attenuating the hemodynamic side effects of non-steroidal anti-inflammatory drugs (NSAID's) and cyclooxygenase-2 (COX-2) inhibitors.

Compounds of formula (I) and their pharmaceutically acceptable derivatives may also be useful in the treatment of neurodegenerative diseases and neurodegeneration such as dementia, particularly degenerative dementia (including senile dementia, Alzheimer's disease, Pick's disease, Huntingdon's chorea, Parkinson's disease and Creutzfeldt-Jakob disease, motor neuron disease); vascular dementia (including multi-infarct dementia); as well as dementia associated with intracranial space occupying lesions; trauma; infections and related conditions (including HIV infection); dementia in Parkinson's disease; metabolism; toxins; anoxia and vitamin deficiency; and mild cognitive impairment associated with ageing, particularly Age Associated Memory Impairment. The compounds may also be useful for the treatment of amyotrophic lateral sclerosis (ALS) and neuroinflamation.

Compounds of formula (I) and their pharmaceutically acceptable derivatives may also be useful in neuroprotection and in the treatment of neurodegeneration following stroke, cardiac arrest, pulmonary bypass, traumatic brain injury, spinal cord injury or the like.

Compounds of formula (I) and their pharmaceutically acceptable derivatives may also be useful in the treatment of tinnitus.

Compounds of formula (I) and their pharmaceutically acceptable derivatives may also be useful in the treatment of psychiatric disease for example schizophrenia, depression (which term is used herein to include bipolar depression, unipolar depression, single or recurrent major depressive episodes with or without psychotic features, catatonic features, melancholic features, atypical features or postpartum onset, seasonal affective disorder, dysthymic disorders with early or late onset and with or without atypical features, neurotic depression and social phobia, depression accompanying dementia for example of the Alzheimer's type, schizoaffective disorder or the depressed type, and depressive disorders resulting from general medical conditions including, but not limited to, myocardial infarction, diabetes, miscarriage or abortion, etc), anxiety disorders (including generalised anxiety disorder and social anxiety disorder), panic disorder, agoraphobia, social phobia, obsessive compulsive disorder and post-traumatic stress disorder, memory disorders, including dementia, amnesic disorders and age-associated memory impairment, disorders of eating behaviours, including anorexia nervosa and bulimia nervosa, sexual dysfunction, sleep disorders (including disturbances of circadian rhythm, dyssomnia, insomnia, sleep apnea and narcolepsy), withdrawal from abuse of drugs such as of cocaine, ethanol, nicotine, benzodiazepines, alcohol, caffeine, phencyclidine (phencyclidine-like compounds), opiates (e.g. cannabis, heroin, morphine), amphetamine or amphetamine-related drugs (e.g. dextroamphetamine, methylamphetamine) or a combination thereof.

Compounds of formula (I) and their pharmaceutically acceptable derivatives may also be useful in preventing or reducing dependence on, or preventing or reducing tolerance or reverse tolerance to, a dependence—inducing agent. Examples of dependence inducing agents include opioids (e.g. morphine), CNS depressants (e.g. ethanol), psychostimulants (e.g. cocaine) and nicotine.

Compounds of formula (I) and their pharmaceutically acceptable derivatives may also be useful in the treatment of kidney dysfunction (nephritis, particularly mesangial proliferative glomerulonephritis, nephritic syndrome), liver dysfunction (hepatitis, cirrhosis), gastrointestinal dysfunction (diarrhoea) and colon cancer.

In one embodiment compounds of the invention may bind selectively to the CB2 receptor; such compounds may be particularly useful in treating CB2 receptor mediated diseases.

The term “treatment” or “treating” as used herein includes the treatment of established disorders and also includes the prophylaxis thereof. The term “prophylaxis” is used herein to mean preventing symptoms in an already afflicted subject or preventing recurrance of symptoms in an afflicted subject and is not limited to complete prevention of an afflication.

According to a further aspect of the invention, we provide a compound of formula (I) or a pharmaceutically acceptable derivative thereof for use in human or veterinary medicine.

According to another aspect of the invention, we provide a compound of formula (I) or a pharmaceutically acceptable derivative thereof for use in the treatment of a condition which is mediated by the activity of cannabinoid 2 receptors.

According to a further aspect of the invention, we provide the use of a compound of formula (I) or a pharmaceutically acceptable derivative thereof for the manufacture of a therapeutic agent for the treatment of a condition which is mediated by the activity of cannabinoid 2 receptors. According to a further aspect of the invention, we provide a method of treating a mammal, for example a human suffering from a condition which is mediated by the activity of cannabinoid 2 receptors which comprises administering to said subject a non toxic, therapeutically effective amount of a compound of formula (I) or a pharmaceutically acceptable derivative thereof.

According to a further aspect of the invention we provide a method of treating a mammal, for example a human suffering from an immune disorder, an inflammatory disorder, pain, rheumatoid arthritis, multiple sclerosis, osteoarthritis or osteoporosis which method comprises administering to said subject a non toxic, therapeutically effective amount of a compound of formula (I) or a pharmaceutically acceptable derivative thereof.

According to another aspect of the invention, we provide a compound of formula (I) or a pharmaceutically acceptable derivative thereof for use in the treatment of a condition such as an immune disorder, an inflammatory disorder, pain, rheumatoid arthritis, multiple sclerosis, osteoarthritis or osteoporosis.

According to another aspect of the invention is provided the use of a compound of formula (I) or a pharmaceutically acceptable derivative thereof for the manufacture of a therapeutic agent for the treatment or prevention of a condition such as an immune disorder, an inflammatory disorder, pain, rheumatoid arthritis, multiple sclerosis, osteoarthritis or osteoporosis.

In one embodiment the condition is pain. In a further embodiment pain is selected from inflammatory pain, viseral pain, cancer pain, neuropathic pain, lower back pain, muscular sceletal, post operative pain, acute pain and migraine. For example, the inflammatory pain is pain associated with rheumatoid arthritis or osteoarthritis.

In order to use a compound of formula (I) or a pharmaceutically acceptable derivative thereof for the treatment of humans and other mammals it is normally formulated in accordance with standard pharmaceutical practice as a pharmaceutical composition. Therefore in another aspect of the invention there is provided a pharmaceutical composition comprising a compound of formula (I) or a pharmaceutically acceptable derivative thereof adapted for use in human or veterinary medicine. In one embodiment the pharmaceutical composition further comprises a pharmaceutical carrier or diluent thereof.

As used herein, “modulator” means both antagonist, partial or full agonist and inverse agonist. In one embodiment the present modulators are agonists In another embodiment the present modulators are antagonists. In one embodiment the compounds of the present invention are CB2 agonists.

Compounds of formula (I) and their pharmaceutically acceptable derivatives may be administered in a standard manner for the treatment of the indicated diseases, for example orally, parentarally, sub-lingually, dermally, intranasally, transdermally, rectally, via inhalation or via buccal administration.

Compounds of formula (I) and their pharmaceutically acceptable derivatives which are active when given orally can be formulated as liquids, tablets, capsules and lozenges. A liquid formulation will generally consist of a suspension or solution of the compound or salt in a liquid carrier for example, ethanol, olive oil, glycerine, glucose (syrup) or water with a flavouring, suspending, or colouring agent. Where the composition is in the form of a tablet, any pharmaceutical carrier routinely used for preparing solid formulations may be used. Examples of such carriers include magnesium stearate, terra alba, talc, gelatin, acacia, stearic acid, starch, lactose and sucrose. Where the composition is in the form of a capsule, any routine encapsulation is suitable, for example using the aforementioned carriers or a semi solid e.g. mono di-glycerides of capric acid, Gelucire™ and Labrasol™, or a hard capsule shell e.g gelatin. Where the composition is in the form of a soft shell capsule e.g. gelatin, any pharmaceutical carrier routinely used for preparing dispersions or suspensions may be considered, for example aqueous gums or oils, and are incorporated in a soft capsule shell.

Typical parenteral compositions consist of a solution or suspension of a compound or derivative in a sterile aqueous or non-aqueous carrier optionally containing a parenterally acceptable oil, for example polyethylene glycol, polyvinylpyrrolidone, lecithin, arachis oil or sesame oil.

Typical compositions for inhalation are in the form of a solution, suspension or emulsion that may be administered as a dry powder or in the form of an aerosol using a conventional propellant such as dichlorodifluoromethane or trichlorofluoromethane.

A typical suppository formulation comprises a compound of formula (I) or a pharmaceutically acceptable derivative thereof which is active when administered in this way, with a binding and/or lubricating agent, for example polymeric glycols, gelatins, cocoa-butter or other low melting vegetable waxes or fats or their synthetic analogs.

Typical dermal and transdermal formulations comprise a conventional aqueous or non-aqueous vehicle, for example a cream, ointment, lotion or paste or are in the form of a medicated plaster, patch or membrane.

In one embodiment the composition is in unit dosage form, for example a tablet, capsule or metered aerosol dose, so that the patient may administer a single dose.

Each dosage unit for oral administration contains suitably from 0.001 mg to 500 mg, for example 0.01 mg to 500 mg such as from 0.01 mg to 100 mg, and each dosage unit for parenteral administration contains suitably from 0.001 mg to 100 mg, of a compound of formula (I) or a pharmaceutically acceptable derivative thereof calculated as the free acid (underivatised compound). Each dosage unit for suppository administration contains suitably from 0.001 mg to 500 mg, for example 0.01 mg to 500 mg such as from 0.01 mg to 100 mg. Each dosage unit for intranasal administration contains suitably 1-400 mg and suitably 10 to 200 mg per person. A topical formulation contains suitably 0.01 to 5.0% of a compound of formula (I).

The daily dosage regimen for oral administration is suitably about 0.01 mg/Kg to 1000 mg/Kg, of a compound of formula(I) or a pharmaceutically acceptable derivative thereof calculated as the free acid (underivatised compound). The daily dosage regimen for parenteral administration is suitably about 0.001 mg/Kg to 200 mg/Kg, of a compound of formula (I) or a pharmaceutically acceptable derivative thereof calculated as the free acid (underivatised compound). The daily dosage regimen for suppository administration is suitably about 0.01 mg/Kg to 1000 mg/Kg, of a compound of formula(I) or a pharmaceutically acceptable derivative thereof calculated as the free acid (underivatised compound). The daily dosage regimen for intranasal administration and oral inhalation is suitably about 10 to about 500 mg/person. The active ingredient may be administered from 1 to 6 times a day, sufficient to exhibit the desired activity.

It may be advantageous to prepare the compounds of the present invention as nanoparticles. This may improve the oral bioavailability of the compounds. For the purposes of the present invention “nanoparticulate” is defined as solid particles with 50% of the particles having a particle size of less than 1 μm, for example less than 0.75 μm

The particle size of the solid particles of compound (I) may be determined by laser diffraction. A suitable machine for determining particle size by laser diffraction is a Lecotrac laser particle size analyser, using an HELOS optical bench fitted with a QUIXEL dispersion unit.

Numerous processes for the synthesis of solid particles in nanoparticulate form are known. Typically these processes involve a milling process, for example a wet milling process in the presence of a surface modifying agent that inhibits aggregation and/or crystal growth of the nanoparticles once created. Alternatively these processes may involve a precipitation process, for example, a process of precipitation in an aqueous medium from a solution of the drug in a non-aqueous solvent.

Accordingly, in a further aspect, the present invention provides a process for preparing compounds of formula (I) and their pharmaceutically acceptable derivatives in nanoparticulate form as hereinbefore defined, which process comprises milling or precipitation.

Representative processes for the preparation of solid particles in nanoparticulate form are described in the patents and publications listed below.

U.S. Pat. No. 4,826,689 to Violanto & Fischer, U.S. Pat. No. 5,145,684 to Liversidge et al U.S. Pat. No. 5,298,262 to Na & Rajagopalan, U.S. Pat. No. 5,302,401 Liversidge et al U.S. Pat. No. 5,336,507 to Na & Rajagopalan, U.S. Pat. No. 5,340,564 to Illig & Sarpotdar U.S. Pat. No. 5,346,702 to Na Rajagopalan, U.S. Pat. No. 5,352,459 to Hollister et al U.S. Pat. No. 5,354,560 to Lovrecich, U.S. Pat. No. 5,384,124 to Courteille et al, U.S. Pat. No. 5,429,824 to June, U.S. Pat. No. 5,503,723 to Ruddy et al, U.S. Pat. No. 5,510 118 to Bosch et al, U.S. Pat. No. 5,518 to Bruno et al, U.S. Pat. No. 5,518,738 to Eickhoff et al, U.S. Pat. No. 5,534,270 to De Castro, U.S. Pat. No. 5,536,508 to Canal et al, U.S. Pat. No. 5,552,160 to Liversidge et al, U.S. Pat. No. 5,560,931 to Eickhoff et al, U.S. Pat. No. 5,560,932 to Bagchi et al, U.S. Pat. No. 5,565,188 to Wong et al, U.S. Pat. No. 5,571,536 to Eickhoff et al, U.S. Pat. No. 5,573,783 to Desieno & Stetsko, U.S. Pat. No. 5,580,579 to Ruddy et al, U.S. Pat. No. 5,585,108 to Ruddy et al, U.S. Pat. No. 5,587,143 to Wong, U.S. Pat. No. 5,591,456 to Franson et al, U.S. Pat. No. 5,622,938 to Wong, U.S. Pat. No 5,662,883 to Bagchi et al, U.S. Pat. No. 5,665,331 to Bagchi et al, U.S. Pat. No. 5,718,919 to Ruddy et al, U.S. Pat. No. 5,747,001 to Wiedmann et al, WO93/25190, WO96/24336, WO97/14407, WO 98/35666, WO99/65469, WO00/18374, WO00/27369, WO00/30615 and WO01/41760.

Such processes may be readily adapted for the preparation of compounds of formula (I) and their pharmaceutically acceptable derivatives in nanoparticulate form. Such processes form a further aspect of the invention.

The process of the present invention may use a wet milling step carried out in a mill such as a dispersion mill in order to produce a nanoparticulate form of the compound. The present invention may be put into practice using a conventional wet milling technique, such as that described in Lachman et al., The Theory and Practice of Industrial Pharmacy, Chapter 2, “Milling” p.45 (1986).

In a further refinement, WO02/00196 (SmithKline Beecham plc) describes a wet milling procedure using a mill in which at least some of the surfaces are made of nylon (polyamide) comprising one or more internal lubricants, for use in the preparation of solid particles of a drug substance in nanoparticulate form.

In another aspect the present invention provides a process for preparing compounds of the invention in nanoparticulate form comprising wet milling a suspension of compound in a mill having at least one chamber and agitation means, said chamber(s) and/or said agitation means comprising a lubricated nylon, as described in WO02/00196.

The suspension of a compound of the invention for use in the wet milling is typically a liquid suspension of the coarse compound in a liquid medium. By “suspension” is meant that the compound is essentially insoluble in the liquid medium. Representative liquid media include an aqueous medium. Using the process of the present invention the average particle size of coarse compound of the invention may be up to 1 mm in diameter. This advantageously avoids the need to pre-process the compound.

In a further aspect of the invention the aqueous medium to be subjected to the milling comprises a compound of formula (I) or a pharmaceutically acceptable derivative thereof present in from about 1% to about 4% w/w, suicabiy from about 10% to about 30% w/w, for example about 20% w/w.

The aqueous medium may further comprise one or more pharmaceutically acceptable water-soluble carriers which are suitable for steric stabilisation and the subsequent processing of compound of formula (I) or a pharmaceutically acceptable derivative thereof after milling to a pharmaceutical composition, e.g. by spray drying. Pharmaceutically acceptable excipients most suitable for steric stabilisation and spray-drying are surfactants such as poloxamers, sodium lauryl sulphate and polysorbates etc; stabilisers such as celluloses e.g. hydroxypropylmethyl cellulose; and carriers such as carbohydrates e.g. mannitol.

In a further aspect of the invention the aqueous medium to be subjected to the milling may further comprise hydroxypropylmethyl cellulose (HPMC) present from about 0.1 to about 10% w/w.

The process of the present invention may comprise the subsequent step of drying compound of the invention to yield a powder.

Accordingly, in a further aspect, the present invention provides a process for preparing a pharmaceutical composition containing a compound of the present invention which process comprises producing compound of formula (I) or a pharmaceutically acceptable derivative thereof in nanoparticulate form optionally followed by drying to yield a powder, and optionally admixing with one or more pharmaceutically acceptable carriers or excipients.

A further aspect of the invention is a pharmaceutical composition comprising a compound of formula (I) or a pharmaceutically acceptable deriviate thereof in which the compound of formula (I) or a pharmaceutically acceptable deriviate thereof is present in solid particles in nanoparticulate form, in admixture with one or more pharmaceutically acceptable carriers or excipients.

By “drying” is meant the removal of any water or other liquid vehicle used during the process to keep compound of formula (I) in liquid suspension or solution. This drying step may be any process for drying known in the art, including freeze drying, spray granulation or spray drying. Of these methods spray drying is particularly preferred. All of these techniques are well known in the art. Spray drying/fluid bed granulation of milled compositions is carried out most suitably using a spray dryer such as a Mobile Minor Spray Dryer [Niro, Denmark], or a fluid bed drier, such as those manufactured by Glatt, Germany.

In a further aspect the invention provides a pharmaceutical composition as hereinbefore defined, in the form of a dried powder, obtainable by wet milling solid particles of compound of formaula (I) followed by spray-drying the resultant suspension.

In one embodiment, the pharmaceutical composition as hereinbefore defined, further comprises HPMC present in less than 15% w/w, for example, in the range 0.1 to 10% w/w.

The CB2 receptor compounds for use in the instant invention may be used in combination with other therapeutic agents, for example COX-2 inhibitors, such as celecoxib, deracoxib, rofecoxib, valdecoxib, parecoxib or COX-189; 5-lipoxygenase inhibitors; NSAID's, such as aspirin, diclofenac, indomethacin, nabumetone or ibuprofen; leukotriene receptor antagonists; DMARD's such as methotrexate; adenosine A1 receptor agonists; sodium channel blockers, such as lamotrigine; NMDA receptor modulators, such as glycine receptor antagonists; gabapentin and related compounds; tricyclic antidepressants such as amitriptyline; neurone stabilising antiepileptic drugs; mono-aminergic uptake inhibitors such as venlafaxine; opioid analgesics; local anaesthetics; 5HT₁ agonists, such as triptans, for example sumatriptan, naratriptan, zolmitriptan, eletriptan, frovatriptan, almotriptan or rizatriptan; EP₁ receptor ligands, EP₄ receptor ligands; EP₂ receptor ligands; EP₃ receptor ligands; EP₄ antagonists; EP₂ antagonists and EP₃ antagonists; bradykinin receptor ligands and vanilloid receptor ligand, antirheumatoid arthritis drugs, for example anti TNF drugs e.g. enbrel, remicade, anti-IL-1 drugs, DMARDS e.g. leflunamide or 5HT₆ compounds. When the compounds are used in combination with other therapeutic agents, the compounds may be administered either sequentially or simultaneously by any convenient route.

Additional COX-2 inhibitors are disclosed in U.S. Pat. Nos. 5,474,995 U.S. Pat. No. 5,633,272; U.S. Pat. No. 5,466,823, U.S. Pat. No. 6,310,099 and U.S. Pat. No. 6,291,523; and in WO96/25405, WO97/38986, WO98/03484, WO97/14691, WO99/12930, WO00/26216, WO00/52008, WO00/38311, WO00/58881 and WO02/18374.

Suitable 5HT6 compounds for a combination suitable for the treatment of e.g Alzhemiers disease or cognative enhancement, may be selected from SGS518 (Saegis), BGC20 761 (BTG disclosed in WO00/34242), WAY466 (Wyeth), PO4368554 (Hoffman le Roche), BVT5182 (Biovitron) and LY483518 (Lily), SB742457 (GSK) and/or compounds disclosed as Example 1 to 50 in WO03/080580.

The compound of the present invention may be administered in combination with other active substances such as 5HT3 antagonists, NK-1 antagonists, serotonin agonists, selective serotonin reuptake inhibitors (SSRI), noradrenaline re-uptake inhibitors (SNRI), tricyclic antidepressants and/or dopaminergic antidepressants.

Suitable 5HT3 antagonists which may be used in combination of the compound of the inventions include for example ondansetron, granisetron, metoclopramide.

Suitable serotonin agonists which may be used in combination with the compound of the invention include sumatriptan, rauwolscine, yohimbine, metoclopramide.

Suitable SSRIs which may be used in combination with the compound of the invention include fluoxetine, citalopram, femoxetine, fluvoxamine, paroxetine, indalpine, sertraline, zimeldine.

Suitable SNRIs which may be used in combination with the compound of the invention include venlafaxine and reboxetine.

Suitable tricyclic antidepressants which may be used in combination with a compound of the invention include imipramine, amitriptiline, chlomipramine and nortriptiline.

Suitable dopaminergic antidepressants which may be used in combination with a compound of the invention include bupropion and amineptine.

Compounds of the present invention may used in combination with PDE4 inhibitors. The PDE4 inhibitor useful in this invention may be any compound that is known to inhibit the PDE4 enzyme or which is discovered to act in as PDE4 inhibitor, and which is only or essentially only a PDE4 inhibitor, not compounds which inhibit to a degree of exhibiting a therapeutic effect other members of the PDE family as well as PDE4. Generally it is preferred to use a PDE4 antagonist which has an IC₅₀ ratio of about 0.1 or greater as regards the IC₅₀ for the PDE4 catalytic form which binds rolipram with a high affinity divided by the IC₅₀ for the form which binds rolipram with a low affinity. Compounds of the present invention or combinations with PDE4 can be used in treating inflammation and as bronchodilators.

There are at least two binding forms on human monocyte recombinant PDE 4 (hPDE 4) at which inhibitors bind. One explanation for these observations is that hPDE 4 exists in two distinct forms. One binds the likes of rolipram and denbufylline with a high affinity while the other binds these compounds with a low affinity. The preferred PDE4 inhibitors of for use in this invention will be those compounds which have a salutary therapeutic ratio, i.e., compounds which preferentially inhibit cAMP catalytic activity where the enzyme is in the form that binds rolipram with a low affinity, thereby reducing the side effects which apparently are linked to inhibiting the form which binds rolipram with a high affinity. Another way to state this is that the preferred compounds will have an IC₅₀ ratio of about 0.1 or greater as regards the IC₅₀ for the PDE 4 catalytic form which binds rolipram with a high affinity divided by the IC₅₀ for the form which binds rolipram with a low affinity.

Reference is made to U.S. Pat. No. 5,998,428, which describes these methods in more detail. It is incorporated herein in full as though set forth herein.

Suitably the PDE4 inhibitors are those PDE4 inhibitors which have an IC₅₀ ratio of greater than 0.5. and particularly those compounds having a ratio of greater than 1.0.

A further aspect of the invention is a CB2 modulator (a compound of formula (I) and their pharmaceutically acceptable derivatives) in combination with a PDE4 inhibitor and pharmaceutical compositions comprising said combination.

A further aspect of the invention is a method of treating lung disorders for example asthma, bronchitis, emphysema, allergic rhinitis, respiratory distress syndrome, pigeon fancier's disease, farmer's lung, chronic obstructive pulmonary disease, (COPD) and cough or a disorder which can be treated with a broncodilator which comprises administering to a mammal including man, an effective amount of a CB2 modulator or a pharmaceutically acceptable derivative thereof (compounds of formula (I) and their pharmaceutically acceptable derivatives) and an effective amount of a PDE4 inhibitor or a pharmaceutically acceptable derivative thereof.

An additional aspect of the invention is the use of an effective amount of a CB2 modulator or a pharmaceutically acceptable derivative thereof (compounds of formula (I) and their pharmaceutically acceptable derivatives) and an effective amount of a PDE4 inhibitor or a pharmaceutically acceptable derivative thereof in the manufacture of a medicament in the treatment of lung disorders for example asthma, bronchitis, emphysema, allergic rhinitis, respiratory distress syndrome, pigeon fancier's disease, farmer's lung, chronic obstructive pulmonary disease, (COPD) and cough or for the manufacture of a brocodilator.

When used herein cough can have a number of forms and includes productive, non-productive, hyper-reactive, asthma and COPD associated.

A further aspect of the invention is a patient pack comprising an effective amount of a CB2 modulator or a pharmaceutically acceptable derivative thereof (compounds of formula (I) and their pharmaceutically acceptable derivatives) and an effective amount of a PDE4 inhibitor or a pharmaceutically acceptable derivative

Possible PDE4 compounds are cis [cyano-4-(3-cyclopentyloxy-4-Methoxyphenyl)cyclohexan-1-carboxylate] also known as cilomilast or Ariflo®, 2-carbomethoxy-4-cyano-4-(3-cyclopropylmethoxy-4-difluoromethoxyphenyl)cyclohexan-1-one, and cis [4-cyano-4-(3-cyclopropylmethoxy-4-difluoromethoxyphenyl)cyclohexan-1-ol]. They can be made by the processed described in U.S. Pat. Nos. 5,449,686 and 5,552,438. Other PDE4 inhibitors, specific inhibitors, which can be used in this invention are AWD-12-281 from ASTA MEDICA (Hofgen, N. et al. 15th EFMC Int Symp Med Chem (September 6-10, Edinburgh) 1998, Abst P.98); a 9-benzyladenine derivative nominated NCS-613 (INSERM); D-4418 from Chiroscience and Schering-Plough; a benzodiazepine PDE4 inhibitor identified as CI-1018 (PD-168787; Parke-Davis/Warner-Lambert); a benzodioxole derivative Kyowa Hakko disclosed in WO 9916766; V-11294A from Napp (Landells, L. J. et al. Eur Resp J [Annu Cong Eur Resp Soc (September 19-23, Geneva) 1998 ]1998, 12(Suppl. 28): Abst P2393); roflumilast (CAS reference No 162401-32-3) and a pthalazinone (WO99/47505) from Byk-Gulden (now Altana); or a compound identified as T-440 (Tanabe Seiyaku; Fuji, K. et al. J Pharmacol Exp Ther,1998, 284(1): 162).

Additional PDE4 inhibitors are disclosed on pages 2 to 15 of WO01/13953. Specifically selected are arofylline, atizoram, BAY-19-8004, benafentrine, BYK-33043, CC-3052, CDP-840, cipamfylline, CP-220629, CP-293121, D-22888, D-4396, denbufylline, filaminast, GW-3600, ibudilast, KF-17625, KS-506-G, laprafylline, NA-0226A, NA-23063A, ORG-20241, ORG-30029, PDB-093, pentoxifylline, piclamilast, rolipram, RPR-117658, RPR-122818, RPR-132294, RPR-132703, RS-17597, RS-25344-000, SB-207499, SB210667, SB211572, SB-211600, SB212066, SB212179, SDZ-ISQ-844, SDZ-MNS-949, SKF-107806, SQ-20006, T-2585, tibenelast, tolafentrine, UCB-29646, V-11294A, YM-58997, YM-976 and zardaverine.

In one embodiment the PDE4 inhibitor is selected from cilomilast, AWD-12-281, NCS-613, D-4418, CI-1018, V-11294A, roflumilast or T-440.

Compounds of the present invention may also be of use in treating atherosclerosis in combination with an anti-hyperlipidaemic, anti-atherosclerotic, anti-diabetic, anti-anginal, anti-hypertension agent or an agent for lowering Lp(a). Examples of the above include cholesterol synthesis inhibitors such as statins, anti-oxidants such as probucol, insulin sensitisers, calcium channel antagonists. Examples of agents for lowering Lp(a) include the aminophosphonates described in WO97/02037, WO98/28310, WO98/28311 and WO98/28312 (Symphar SA and SmithKline Beecham). Examples of antihyerpertension agents are angiotensin-converting enzyme inhibitors, angiotensin-II receptor antagonists, ACE/NEP inhibitors, -blockers, calcium channel blockers, PDE inhibitors, aldosterone blockers

A possible combination therapy will be the use of a compound of the present invention and a statin. The statins are a well known class of cholesterol lowering agents and include atorvastatin, simvarstatin, pravastatin, cerivastatin, fluvastatin, lovastatin and ZD 4522 (also referred to as S-4522, Astra Zeneca). The two agents may be administered at substantially the same time or at different times, according to the discretion of the physician.

A further possible combination therapy will be the use of a compound of the present invention and an anti-diabetic agent or an insulin sensitiser. Within this class, possible compounds for use with a compound of the present invention include the PPARgamma activators, for instance G1262570 (Glaxo Wellcome) and also the glitazone class of compounds such as rosiglitazone (Avandia, SmithKline Beecham), troglitazone and pioglitazone.

It will be appreciated that the compounds of any of the above combinations or compositions may be administered simultaneously (either in the same or different pharmaceutical formulations), separately or sequentially.

The invention thus provides, in a further aspect, a combination comprising a compound of formula (I) or a pharmaceutically acceptable derivative thereof together with a further therapeutic agent or agents.

The combinations referred to above may conveniently be presented for use in the form of a pharmaceutical formulation and thus pharmaceutical formulations comprising a combination as defined above together with a pharmaceutically acceptable carrier or excipient comprise a further aspect of the invention. The individual components of such combinations may be administered either sequentially or simultaneously in separate or combined pharmaceutical formulations.

When a compound of formula (I) or a pharmaceutically acceptable derivative thereof is used in combination with a second therapeutic agent active against the same disease state the dose of each compound may differ from that when the compound is used alone. Appropriate doses will be readily appreciated by those skilled in the art.

### Determination of Cannabinoid CB1 Receptor Agonist Activity

The cannabinoid CB1 receptor agonist activity of compounds of formula (I) was determined in accordance with the following experimental method.

### Experimental Method

Yeast (Saccharomyces cerevisiae) cells expressing the human cannabinoid CB1 receptor were generated by integration of an expression cassette into the ura3 chromosomal locus of yeast strain MMY23. This cassette consisted of DNA sequence encoding the human CB1 receptor flanked by the yeast GPD promoter to the 5′ end of CB1 and a yeast transcriptional terminator sequence to the 3′ end of CB1. MMY23 expresses a yeast/mammalian chimeric G-protein alpha subunit in which the C-terminal 5 amino acids of Gpa1 are replaced with the C-terminal 5 amino acids of human Gαi½ (as described in Brown et al. (2000), Yeast 16:11-22). Cells were grown at 30° C. in liquid Synthetic Complete (SC) yeast media (Guthrie and Fink (1991), Methods in Enzymology, Vol. 194) lacking uracil, tryptophan, adenine and leucine to late logarithmic phase (approximately 6 OD₆₀₀/ml).

Agonists were prepared as 10 mM stocks in DMSO. EC₅₀ values (the concentration required to produce 50% maximal response) were estimated using 4 fold dilutions (BiomekFX, Beckman) into DMSO. Agonist solutions in DMSO (1% final assay volume) were transferred into black microtitre plates from Greiner (384-well). Cells were suspended at a density of 0.2 OD₆₀₀/ml in SC media lacking histidine, uracil, tryptophan, adenine and leucine and supplemented with 10 mM 3-aminotriazole, 0.1M sodium phosphate pH 7.0, and 10 μM fluorescein di-β-D-glucopyranoside (FDGlu). This mixture (50 l per well) was added to agonist in the assay plates (Multidrop 384, Labsystems). After incubation at 30° C. for 24 hours, fluorescence resulting from degradation of FDGlu to fluorescein due to exoglucanase, an endogenous yeast enzyme produced during agonist-stimulated cell growth, was determined using a fluorescence microtitre plate reader (Tecan Spectrofluor or LJL analyst excitation wavelength: 485 nm; emission wavelength: 535 nm). Fluorescence was plotted against compound concentration and iteratively curve fitted using a four parameter fit to generate a concentration effect value. Efficacy (Eₘₐₓ) was calculated from the equation

Eₘₐₓ=Max_{[compound X]}−Min_{[compound X]}/Max_{[HU210]}−Min_{HU210]}×100%

where Max_{[compound X]} and Min_{[compound X]} are the fitted maximum and minimum respectively from the concentration effect curve for compound X, and Max_{[HU210]} and Min_{[HU210]} are the fitted maximum and minimum respectively from the concentration effect curve for (6aR,10aR)-3-(1,1′-Dimethylheptyl)-6a,7,10,10a-tetrahydro-1-hydroxy-6,6-dimethyl-6H-dibenzo[b,d]pyran-9-methanol (HU210; available from Tocris). Equieffective molar ratio (EMR) values were calculated from the equation

EMR=EC_{50[compound X]}/EC_{50[HU210]}

Where EC_{50[compound X]} is the EC₅₀ of compound X and EC_{50[HU210]} is the EC₅₀ of HU210.

The compounds of Examples 1 to 22 were tested according to this method and had EC₅₀ values >1,000 nM and/or an efficacy of <30% at the cloned human cannabinoid CB1 receptor. The results given are averages of a number of experiments.

### Determination of Cannabinoid CB2 Receptor Agonist Activity

The cannabinoid CB2 receptor agonist activity of compounds of formula (I) was determined in accordance with the following experimental method.

### Experimental Method

Yeast (Saccharomyces cerevisiae) cells expressing the human cannabinoid CB2 receptor were generated by integration of an expression cassette into the ura3 chromosomal locus of yeast strain MMY23. This cassette consisted of DNA sequence encoding the human CB2 receptor flanked by the yeast GPD promoter to the 5′ end of CB2 and a yeast transcriptional terminator sequence to the 3′ end of CB2. MMY23 expresses a yeast/mammalian chimeric G-protein alpha subunit in which the C-terminal 5 amino acids of Gpa1 are replaced with the C-terminal 5 amino acids of human Gαi½ (as described in Brown et al. (2000), Yeast 16:11-22). Cells were grown at 30° C. in liquid Synthetic Complete (SC) yeast media (Guthrie and Fink (1991), Methods in Enzymology, Vol. 194) lacking uracil, tryptophan, adenine and leucine to late logarithmic phase (approximately 6 OD₆₀₀/ml).

Agonists were prepared as 10 MM solutions in DMSO. EC₅₀ values (the concentration required to produce 50% maximal response) were estimated using 4 fold dilutions (BiomekFX, Beckman) into DMSO. Agonist solutions in DMSO (1% final assay volume) were transferred into black microtitre plates from Greiner (384-well). Cells were suspended at a density of 0.2 OD₆₀₀/ml in SC media lacking histidine, uracil, tryptophan, adenine and leucine and supplemented with 10 mM 3-aminotriazole, 0.1M sodium phosphate pH 7.0, and 10 μM fluorescein di-β-D-glucopyranoside (FDGlu). This mixture (50 μl per well) was added to agonist in the assay plates (Multidrop 384, Labsystems). After incubation at 30° C. for 24 hours, fluorescence resulting from degradation of FDGlu to fluorescein due to exoglucanase, an endogenous yeast enzyme produced during agonist-stimulated cell growth, was determined using a fluorescence microtitre plate reader (Tecan Spectrofluor or LJL Analyst excitation wavelength: 485 nm; emission wavelength: 535 nm). Fluorescence was plotted against compound concentration and iteratively curve fitted using a four parameter fit to generate a concentration effect value. Efficacy (Eₘₐₓ) was calculated from the equation

Eₘₐₓ=Max_{[compound X]}−Min_{[compound X]}/Max_{[HU210]}−Min_{[HU210]}×100%

where Max_{[compound X]} and Min_{[compound X]} are the fitted maximum and minimum respectively from the concentration effect curve for compound X, and Max_{[HU210]} and Min_{[HU210]} are the fitted maximum and minimum respectively from the concentration effect curve for (6aR,10aR)-3-(1,1′-Dimethylheptyl)-6a,7,10,10a-tetrahydro-1-hydroxy-6,6-dimethyl-6H-dibenzo [b,d]pyran-9-methanol (HU210; available from Tocris). Equieffective molar ratio (EMR) values were calculated from the equation

EMR=EC_{50[compound X]}/EC_{50[HU210]}

Where EC50[compound X] is the EC₅₀ of compound X and EC_{50[HU210]} is the EC₅₀of HU210.

The compounds of Examples 1 to 22 were tested according to this method and had EC₅₀ values of <300 nM and efficacy value of >50% at the cloned human cannabinoid CB2 receptor. The results given are averages of a number of experiments.

The compounds of Examples 1 to 22 tested according to the above methods had an EMR of greater than 100 in the CB1 yeast receptor assay and an EMR of less than 100 in the CB2 yeast receptor assay. Compounds of Examples 1-5, and 7-22 had at least a tenfold lower EMR for CB2 over CB1. The results given are averages of a number of experiments.

### Measurement of CB2 Agonist Effects in a Reporter Gene Assay

### Experimental Method

CB2 agonist effects were determined using a reporter gene assay. These studies were performed using a CHO-K1 cell line expressing human recombinant CB2 receptors (CHO-K1 CB2 CRE-LUC cells). These cells additionally express a “CRE-LUC” reporter gene construct comprising the gene for luciferase under the control of multiple cAMP response element binding protein promoters. In these cells, increases in intracellular cAMP levels leads to transcription of the luciferase gene and the subsequent production of luciferase. The expression of luciferase is measured by addition to the cells of a proprietary mixture containing luciferin, the substrate for luciferase (Luclite, Perkin Elmer, Cat No 6016919). The resultant reaction leads to the generation of light which is measured in a TopCount scintillation counter. In the CHO-K1 CB2 CRE-LUC cells, forskolin produces a marked increase in luciferase expression and CB2 agonists inhibit this response. The CHO-K1 CB2 CRE-LUC cells routinely express a high level of constitutive CB2 receptor activity. This was overcome in these experiments by pre-treating the cells with the inverse agonist, SR144528, for 30-60mins before use. This treatment has been shown to eliminate constitutive CB2 receptor activity (Bouaboula et al., 1999).

### Methods

CHO-K1 CB2 CRE-LUC cells were grown in DMEM/F12 plus glutamax I medium (Gibco Cat. No. 31331-028), supplemented with 9% FBS (Gibco, Cat. No. 16000-040) and 0.5 mg.ml⁻¹ G418 (Gibco, Cat. No. 10131-027) and 0.5 mg.ml⁻¹ Hygromycin (Invitrogen, Cat. No. 10687-010). Cells were grown as a monolayer culture in 162 cm² vented Nunclon flasks (NUNC, Cat. No. 178883) in 27.5 ml of media in a humidified 95% air and 5% CO₂ atmosphere at 37° C. When confluent, the growth media was replaced with DMEM/F12 medium (Gibco, Cat. No. 31331-028) containing 100 nM of the CB2 inverse agonist, SR144528, and the cells were incubated at 37° C. for 30-60 mins. Flasks were rinsed twice with 25 ml Dulbecco's phosphate buffered saline (PBS, Gibco Cat. No. 14190-094) and then harvested by incubation for 10 mins in 10 ml of Versene (Gibco, Cat. No. 15040-033). Cells were detached by a sharp blow to the flask and the cell suspension made up to 50 ml with PBS and centrifuged at 250×g for 5mins. The cell pellet was re-suspended in 24 mls of phenol-red free DMEM/F12 assay buffer (Gibco, Cat. No. 11039-021) and 50 μl of cell suspension (approximately 50,000 cells) added to 96 well plates (Costar, Cat. No. 3904—clear bottomed black well plates) containing 50 μl of test agonist in 2 μM forskolin (final assay concentration of 1 μM FSK). Test agonists were prepared as 10 mM solutions in DMSO and diluted into phenol-red free DMEM/F12 assay buffer containing 2 μM forskolin to produce a 20 μM solution of test agonist. Subsequent serial dilutions of test agonist were prepared in the assay buffer containing forskolin and each test agonist was routinely examined over a final assay concentration range of 10 μM to 10 nM (or lower if required). The plates were mixed on a plate shaker for 5 mins (800-1000 rpm) and then centrifuged briefly (5-10 s) at 250×g, placed in a Bioplate without their lids, and incubated for 4-5 hr in a humidified 95% air and 5% CO₂ atmosphere at 37° C. The 96 well plates were removed from the incubator and placed at RT for 10-15 mins before addition of 25 μl of Luclite solution, prepared according to the manufacturer's instructions. The plates were sealed with Topseal A (Perkin Elmer, Cat. No. 6005185), mixed on a plate shaker for 5 mins (800-1000 rpm) and then centrifuged briefly (5-10 s) at 250×g. Finally, luminescence was measured using a Packard TopCount scintillation counter.

### Data Analysis

For each compound maximal inhibition of the forsklin response and the EC50 for this effect was determined. In each experiment the reference agonist HU210 was included and the maximal effect of each test agonist was expressed relative to the maximal effect produced by HU210 to provide an estimate of intrinsic activity. In addition the EC50 of each compound was divided by the EC50 for HU210 to calculate the equipotent molar ratio (EMR) for the test compound.

### Results

Compounds of examples 1-5, 9-10, 17 and 20 tested according to this method and had EMR values of less than 30. The results given are averages of a number of experiments.

### REFERENCE

Bouaboula M. Dussossoy D. Casellas P. Regulation of peripheral cannabinoid receptor CB2 phosphorylation by the inverse agonist SR 144528. Implications for receptor biological responses. Journal of Biological Chemistry. 274(29):20397-405, 1999

The following examples are illustrative, but not limiting of the embodiments of the present invention.

### Abbreviations:

AcOH (acetic acid), Bn (benzyl), Bu, Pr, Me, Et (butyl, propyl, methyl ethyl), DMSO (dimethyl sulfoxide), DCM (dichloromethane), DME (1,2-dimethoxyethane), DMF (N,N-dimethylformamide), EDC (1-(3-dimethylaminopropyl)-3-ethylcarbodiimide), EtOAc (ethyl acetate), EtOH (ethanol), HPLC (High pressure liquid chromatography), LC/MS (Liquid chromatography/Mass spectroscopy), MDAP (Mass Directed AutoPurification), MeCN (acetonitrile), MeOH (methanol), NMR (Nuclear Magnetic Resonance (spectrum)), NMP (N-methyl pyrrolidone), SCX (strong cation exchanger e.g. Isolute SCX-2 cartridges), SPE (Solid Phase Extraction), TFA (Trifluoroacetic acid), THF (tetrahydrofuran), s, d, t, q, m, br (singlet, doublet, triplet, quartet, multiplet, broad.)

### Hardware

Waters 2525 Binary Gradient Module
Waters 515 Makeup Pump
Waters Pump Control Module
Waters 2767 Inject Collect
Waters Column Fluidics Manager
Waters 2996 Photodiode Array Dectector
Waters ZQ Mass Spectrometer
Gilson 202 fraction collector
Gilson Aspec waste collector

### Software

Waters Masslynx version 4 SP2

### Column

The columns used are Waters Atlantis, the dimensions of which are 19 mm×100 mm (small scale) and 30 mm×100 mm (large scale). The stationary phase particle size is 5 μm.

### Solvents

A: Aqueous solvent=Water+0.1% Formic Acid
B: Organic solvent=Acetonitrile+0.1% Formic Acid
Make up solvent=Methanol: Water 80:20
Needle rinse solvent=Methanol

### Methods

There are four methods used depending on the analytical retention time of the compound of interest. They all have a 13.5-minute runtime, which comprises of a 10-minute gradient followed by a 3.5 minute column flush and re-equilibration step.
Large/Small Scale 1.0-1.5=5-30% B
Large/Small Scale 1.5-2.2=15-55% B
Large/Small Scale 2.2-2.9=30-85% B
Large/Small Scale 2.9-3.6=50-99% B
Large/Small Scale 3.6-5.0=80-99% B (in 6 mins)

### Flow Rate

All of the above methods have a flow rate of either 20 mls/min (Small Scale) or 40 mls/min (Large Scale)

### Analytical LCMS Systems

### Hardware

Agilent 1100 Gradient Pump
Agilent 1100 Autosampler
Agilent 1100 DAD Dectector
Agilent 1100 Degasser
Agilent 1100 Oven
Agilent 1100 Controller
Waters ZQ Mass Spectrometer
Sedere Sedex 75 or Sedere Sedex 85 or Polymer Labs PL-ELS-2100

### Software

Waters MassLynx version 4.0 SP2

### Column

The column used is a Waters Atlantis, the dimensions of which are 4.6 mm×50 mm. The stationary phase particle size is 3 μm.

### Solvents

A: Aqueous solvent=Water+0.05% Formic Acid
B: Organic solvent=Acetonitrile+0.05% Formic Acid

### Method

The generic method used has a 5 minute runtime.

| Time/min | % B |
|---|---|
| 0 | 3 |
| 0.1 | 3 |
| 4 | 97 |
| 4.8 | 97 |
| 4.9 | 3 |
| 5.0 | 3 |

### Flow Rate

The above method has a flow rate of 3 ml/mins

Conditions used for NMR

### Hardware

Bruker 400 MHz Ultrashield
Bruker B-ACS60 Autosampler
Bruker Advance 400 Console
Software
User interface—NMR Kiosk
Controlling software—XWin NMR version 3.0
   Conditions used for the Microwave

### Hardware

Biotage Initiator
Specifications
Heating temperature up to 250° C.
Microwave radiation 50-300 W at 2.45 GHz

### Intermediate 1: Ethyl 6-chloro-4-(methylamino)-5-nitro-3-pyridinecarboxylate

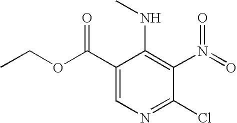

Preparation a: Methylamine (33% in ethanol, 1 mL) was added dropwise to a refluxing solution of ethyl4,6-dichloro-5-nitro-3-pyridinecarboxlate (may be prepared according to Sanchez et al, J. Heterocyclic Chem., 1993, 30, 855) (2.65 g) and triethylamine (1.4 mL) in ethanol (10 mL). The reaction was refluxed for 30 minutes then evaporated. The residue was extracted with boiling ethyl acetate which was then evaporated. The resulting crude product was extracted with boiling hexane which, on cooling, yielded the title compound as yellow crystals (1.82 g) mp 70-72° C.

Preparation b: To a solution of ethyl 4,6-dichloro-5-nitro-3-pyridinecarboxylate (75.96 g, 0.287 moles) in ethanol (596 ml) was added triethylamine (40 ml, 0.287 moles), and the mixture was heated to reflux. Methylamine (35.6 ml, 33%) in ethanol was added drop wise to the refluxing mixture over 1 hour 35 minutes. After complete addition the mixture was refluxed for 25 min and then allowed to cool The reaction mixture was evaporated on a buchi under vacuum. The residue obtained was stirred in DCM (200 ml) for 10 minutes; the solid was filtered off and washed with DCM (100 ml). The DCM layers were combined and extracted with water (2×250 ml). The water layer was re-extracted with DCM (200 ml). The DCM layers were combined, dried using MgSO₄. The MgSO₄ was filtered off and the DCM layer was evaporated to give a reddish-brown oil. This solidifies on standing. The solid was taken up into ethanol (150 ml) and heated until the solid had gone into solution. The mixture was allowed to cool overnight, the crystals formed were filtered off, washed with cold ethanol (100 ml). The crystals were dried in air under vacuum to give ethyl 6-chloro-4-(methylamino)-5-nitro-3-pyridinecarboxylate (52.1 g, 69%)

NMR (400 MHz, DMSO-d6) HNC121277 δ1.40-1.44 (3H, t), 2.92-2.94 (3H, d), 4.37-4.43 (2H, q), 8.73 (1H, s), 9.00-9.10 (1H, br). Consistent with proposed structure

LC/MS Product 3.10 min, [MH⁺]260 consistent with the molecular formula C₉H₁₀N₃ClO₄. 8% of an impurity present at 2.45 min, [MH⁺]255.

### Intermediate 2: Ethyl 5-amino-6-chloro-4-(methylamino)-3-pyridinecarboxylate

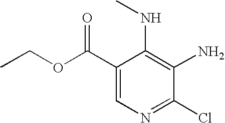

Preparation a: A suspension of ethyl 6-chloro-4-(methylamino)-5-nitro-3-pyridinecarboxylate (15 g) in ethanol was hydrogenated in the presence of Raney nickel at room temperature and atmospheric pressure. After completion, the catalyst was filtered and the filtrate evaporated to give a dark oil. Trituration with hexane yielded the title compound as a dark pink solid (12 g) mp 50-52° C.

Preparation b: To ethyl 6-chloro-4-(methylamino)-5-nitro-3-pyridinecarboxylate (52.1 g, 0.2 moles) was added ethanol (300 ml). To this suspension was added Raney nickel (6 ml of a 50% slurry in water) under argon. The reaction was stirred under hydrogen atmosphere at room temperature overnight (23 hours). The Raney nickel was filtered off using Kieselguhr under argon. The ethanol was evaporated on a buchi under vacuum to give a ethyl 5-amino-6-chloro-4-(methylamino)-3-pyridinecarboxylate (49.7 g 107%) as a thick brown residue. The mixture was taken on without further purification.

NMR (400 MHz, DMSO-d6) HNC121452 δ Consistent within reason to the proposed structure LC/MS Product 2.05 min, [MH⁺]230 . Number of impurities present from 2% to 9%. Product consistent with the molecular formula C₉H₁₀N₃ClO₄

### Intermediate 3: Ethyl 4-chloro-1-methyl-1H-imidazo[4,5-c]pyridine-7-carboxylate

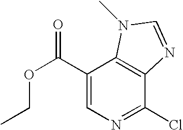

Preparation a: A mixture of ethyl 5-amino-6-chloro-4-(methylamino)-3-pyridinecarboxylate (12 g) and triethylorthoformate (50 mL) was refluxed for three hours (ethanol was removed). The hot solution was filtered then allowed to cool. The resulting solid was filtered and washed with ether then dried to yield the title compound as a brown crystalline solid (8.8 g) mp 112-114° C.

Preparation b: To ethyl 5-amino-6-chloro-4-(methylamino)-3-pyridinecarboxylate (49.7 g, 0.21 moles) was added triethylorthoformate (216 ml, 1.26 moles) and the mixture was heated to reflux for 1 hour. The mixture was allowed to cool and evaporated on a buchi under vacuum to give a thick semi solid. Diethyl ether (500 ml) was added to the semi solid and the mixture was stirred at room temperature for 10 minutes. The brown solid was filtered off and further washed with diethyl ether (250 ml), The solid was dried under vacuum in air to give ethyl 4-chloro-1-methyl-1H-imidazo[4,5-c]pyridine-7-carboxylate (31.7 g, 61%)

NMR (400 MHz, Chloroform-d6) HNC121507 δ1.46-1.49 (3H, t), 4.16 (3H, s), 4.45-4.15 (2H, q), 7.99 (1H, s), 8.78 (1H,s). Consistent with proposed structure

### Intermediate 4: Ethyl 4-[(3-bromophenyl)amino]-1-methyl-1H-imidazo[4,5-c]pyridine-7-carboxylate

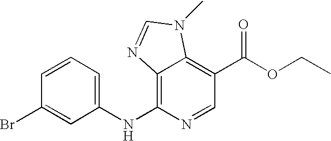

A suspension of ethyl 4-chloro-1-methyl-1H-imidazo[4,5-c]pyridine-7-carboxylate (650 mg) in 1,4-dioxane (5 ml) was prepared in a 20 ml microwave vial. 3-Bromoaniline (935 mg) was added to this, followed by methanesulphonic acid (0.35 ml). The reaction vial was sealed and heated to 180° C. for 30 minutes. At this point the reaction mixture was combined with a batch from another reaction completed in the same manner but using ethyl 4chloro-1-methyl-1H-imidazo[4,5-c]pyridine -7-carboxylate (100 mg). This combined reaction mixture was partitioned between dichloromethane and water and the organic layer collected by passing it through a hydrophobic frit. The dichloromethane solution was reduced in vacuo, and the compound purified by silica chromatography (50 g cartridge, eluting 0-100% ethyl acetate in hexane) to yield the title compound which was dried in vacuo to yield a cream coloured solid (1.1 g)

LC/MS ]MH⁺]377 consistent with molecular formula C₁₆H₁₅⁸¹BrN₄O₂

### Intermediate 5: Sodium 4-[(3-bromophenyl)amino]-1-methyl-1H-imidazo[4,5-c]pyridine-7-carboxylate

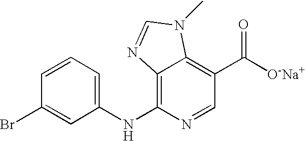

Ethyl-4-[(3-bromophenyl)amino]-1-methyl-1H-imidazo[4,5-c]pyridine-7-carboxylate (1.1 g) was placed in a 20 ml microwave vial and dissolved in methanol (15 ml) then (2N) sodium hydroxide (4 ml) was added. The vial was sealed and heated to 120° C. for 5 minutes. The solution was dried in vacuo to give the title compound as a white solid (8.7 g including excess sodium hydroxide)

LC/MS [MH⁺]349 consistent with molecular formula C₁₄H₁₁⁸¹BrN₄O₂

### Intermediate 6: Ethyl 4-1(2,4-dichlorophenyl)amino]-1-methyl-1H-imidazo[4,5-c]pyridine-7-carboxylate.

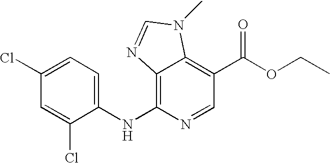

A suspension of ethyl 4-chloro-1-methyl-1H-imidazo[4,5-c]pyridine-7-carboxylate (650 mg) in 1,4-dioxane (5 ml) was made in a 20 ml microwave vial. To this 2,4-dichloroaniline (880 mg) was added followed by methanesulphonic acid (0.35 ml). The reaction vial was sealed and heated to 180° C. for 30 minutes. At this point the reaction mixture was combined with a batch from another reaction completed in the same manner but using 100 mg quantities of ethyl 4-chloro-1-methyl-1H-imidazo[4,5-c]pyridine-7-carboxylate. This combined reaction mixture was partitioned between dichloromethane and water and the organic layer collected by passing it through a hydrophobic frit. The dichloromethane solution was reduced on vacuo. The residue was purified by silica chromatography (50 g cartridge, eluting 0-100% ethyl acetate in hexane), however some precipitate remained after loading onto the column. This was washed with methanol on an SCX cartridge (5 g) and analysed, proving to be the title compound. The correct fraction from purification was dried in vacuo and combined with the precipitate to give a brown solid (700 mg).

LC/MS [MH⁺]365 consistent with molecular formula C₁₆H₁₄³⁵Cl₂N₄O₂

### Intermediate 7: 4-[(2,4-Dichlorophenyl)amino]-1-methyl-1H-imidazo[4,5-c]pyridine-7-carboxylic acid hydrochloride salt.

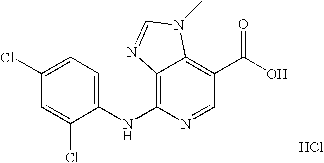

The ethyl 4-[(2,4-dichlorophenyl)amino]-1-methyl-1H-imidazo[4,5-c]pyridine-7-carboxylate (700 mg) was placed in a 20 ml microwave vial and dissolved in methanol (15 ml) then 2N sodium hydroxide was added (4 ml). The vial was sealed and heated to 120° C. for 5 minutes. The solution was reduced it vacuo and re-dissolved in methanol (30 ml). (2N) sodium hydroxide (4 ml) was added and the reaction refluxed for 3 hours at 100° C. The reaction mixture was dried in vacuo and acidified using (2N) hydrochloric acid, the suspension filtered and the solid dried in vacuo to give the title compound (540 mg)

LC/MS [MH⁺]337 consistent with molecular formula C₁₄H₁₀³⁵Cl₂N₄O₂

### Intermediate 8: Ethyl 4-[(3-chlorophenyl)amino]-1-methyl-1H-imidazo[4,5-c]pyridine-7-carboxylate.

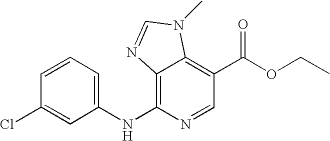

Preparation a: A suspension of ethyl 4-chloro-1-methyl-1H-imidazo[4,5-c]pyridine-7-carboxylate (1 g, 4.1 mmol) and 3-chloroaniline (0.9 ml, 8.9 mmol) in 1,4-dioxane (25 ml) was heated at 100° C. overnight. The crude reaction mixture was evaporated and partitioned between ethyl acetate and water (approx. 100 ml each). The ethyl acetate layer was dried, filtered and evaporated to give the title compound as a crude orange oil (1.8 g).

LC/MS [MH⁺]331 consistent with molecular formula C₁₆H₁₅³⁵ClN₄O₂

Preparation b: To ethyl 4-chloro-1-methyl-1H-imidazo[4,5-c]pyridine-7-carboxylate (31.7 g, 0.13 moles), was added 1,4-dioxan (410 ml), 3- Chloroaniline (27.93 ml, 0.26 moles), and methanesulfonic acid (17.19 ml, 0.26 moles) A small exothermic reaction was noted. The mixture was heated to 105° C. for 4 hours. The dioxane was removed on a buchi under vacuum. To the residue was added ethyl acetate (1 litre) and water (500 ml), this solution was neutralised by addition saturated aqueous sodium bicarbonate (350 ml). The ethyl acetate layer was separated and the aqueous layer was re-extracted with ethyl acetate (500 ml). The ethyl acetate layers were combined and evaporated on a buchi under vacuum. To the residue was added hexane (1.5 litre) and the mixture was heated to reflux for 45 minutes. On cooling the solid obtained was filtered and heated to reflux with an additional amount of hexane (1 litre). On cooling the solid was filtered off to give ethyl 4-[(3-chlorophenyl)amino]-1-methyl-1H-imidazo[4,5-c]pyridine-7-carboxylate (37.9 g, 86%) as a dark brown solid.

NMR (400 MHz, Chloroform-d6) HNC121507 δ1.41-1.44 (3H, t), 4.14 (3H, s), 4.37-4.42 (2H, q), 7.02-7.05 (1H, m), 7.25-7.29 (1H, m), 7.57-7.60 (1H, m), 7.93 (1H, s), 7.80-8.10 (1H, br) 8.12 (1H, s), 8.74 (1H, s). Consistent with proposed structure

LC/MS Product retention time 3.19 min, [MH⁺]331 consistent with the molecular formula C₁₆H₁₅N₄ClO₂

### Intermediate 9: 4-1(3-Chlorophenyl)amino]-1-methyl-1H-imidazo[4,5-c]pyridine-7-carboxylic acid.

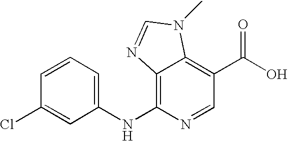

Preparation a: Ethyl 4-[(3-chlorophenyl)amino]-1-methyl-1H-imidazo[4,5-c]pyridine-7-carboxylate (1.8 g) was dissolved into both methanol (5 ml) and (2N) sodium hydroxide (5 ml) and heated under microwave conditions at 120° C. for 5 minutes. The compound was then partitioned between ethyl acetate and water (100 ml). The ethyl acetate layer was dried, filtered and evaporated. The crude material was then dissolved in water and taken to (pH 4-3) with (2N) hydrochloric acid which lead to a precipitate crashing out from the water. Ethyl acetate was added, which caused the mixture to form an emulsion. The whole emulsion was then evaporated and the sample was purified using an amino-propyl SPE cartridge (50 g) eluting with (2M) ammonia in methanol, to afford title compound (1.1 g).

LC/MS [MH⁺]303 consistent with molecular formula C₁₄H₁₁³⁵ClN₄O₂

Preparation b: To ethyl 4-[(3-chlorophenyl)amino]-1-methyl-1H-imidazo[4,5-c]pyridine-7-carboxylate (32.9 g, 0.099 moles) was added ethanol (330 ml) followed by 2M aqueous sodium hydroxide (130 ml, 0.25 moles). The mixture was heated under stirring to reflux for 1 hour. On cooling the mixture set solid, ethanol (100 ml) was added to form slurry. The slurry was evaporated on a buchi under vacuum to give a brown solid. This was taken up into water (1 litre) and the solution was cooled in an ice bath to 15° C., and acidified to pH1 using 2M aqueous hydrochloric acid. The precipitate formed was filtered off, the solid was washed with water (2×200 ml). The solid was dried under vacuum at 40° C. until a constant weight was achieved (48 hours) to give 4-[(3-chlorophenyl)amino]-1-methyl-1H-imidazo[4,5-c]pyridine-7-carboxylic acid (28.1 9 g, 93%) as a brown solid.

NMR (400 MHz, DMSO-d6) HNC121878 δ4.07 (3H, s), 7.04-7.06 (1H, m), 7.31-7.36 (1H, t), 7.92-7.94 (1H, m), 8.23-8.24 (1H, m), 8.33 (1H, s), 8.49 (1H, s), 9.82 (1H, s), 12.00-13.50 (broad signal). Consistent with proposed structure

LC/MS Product retention time 2.17 min, [MH⁺]303 consistent with the molecular formula C₁₄H₁₁N₄ClO₂

### Intermediate 10: 4-[(3-Chlorophenyl)oxy]-1-methyl-1H-imidazo [4,5-c]pyridine-7-carboxylic acid hydrochloride salt.

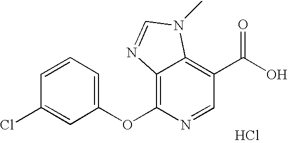

A mixture of 3-chlorophenol (1.8 ml, 16.7 mmol) in 1,4-dioxane (4 ml) was stirred vigorously. Sodium hydride (60% in mineral oil, 701 mg) was then slowly added. More 1,4-dioxane (18 ml) was added to the suspension along with ethyl 4-chloro-1-methyl-1H-imidazo[4,5-c]pyridine-7-carboxylate (1 g, 4.2 mmol). The sample was heated under microwave conditions at 180° C. for 10 hours. The material was then evaporated to as dry as possible, re-dissolved into water and acidified to pH-1 with (2N) hydrochloric acid. A solid precipitate was obtained which was filtered and dried in a vac-oven at 40° C. overnight (1.3 g).

LC/MS [MH⁺]304 consistent with molecular formula C₁₄H₁₀³⁵ClN₃O₃

### Intermediate 11: 4-Chloro-1-methyl-1H-imidazo[4,5-c]pyridine-7-carboxylic acid

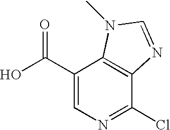

Ethyl 4-chloro-1-methyl-1H-imidazo[4,5-c]pyridine-7-carboxylate (8.80 g), methanol (90 ml) and 2N sodium hydroxide (30 ml) were stirred together at room temperature for two hours. Addition of 2N hydrochloric acid (30 ml) afforded a precipitate which was filtered off and dried under vacuum at 50° C. to yield the title compound as a red powder (6.7 g).

LC/MS [MH⁺]212 consistent with molecular formula C₈H₆³⁵ClN₃O₂

### Intermediate 12: 4-Chloro-1-methyl-7-(4-morpholinylcarbonyl)-1H-imidazo[4,5-c]pyridine

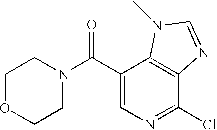

A mixture of 4-chloro-1-methyl-1H-imidazo[4,5-c]pyridine-7-carboxylic acid (1.0 g) in dimethylformamide (30 ml), N,N-diisopropylethylamine (4.12 ml), morpholine (0.82 ml) and O-(1H-benzotriazol-1-yl)-N,N,N′,N′-tetramethyluronium hexafluorophosphate (2.688 g) was stirred at room temperature for forty five minutes. The reaction mixture was dissolved in water and ethyl acetate. The organic layer was washed twice with aqueous saturated sodium hydrogen carbonate, then with water. The organic layer was evaporated, the water washings were evaporated, and the combined sodium bicarbonate washings were evaporated. The residue from evaporation of the sodium bicarbonate washings was stirred in dichloromethane, the solid was filtered off and the filtrate combined with the residues from evaporation of the organic layer and the residue from the water washings. The resultant mixture was evaporated, and the residue was purified by chromatography (50 g C₁₈ column) using a gradient of 0-100% methanol/water to afford the title compound as an off-white solid (940 mg).

LC/MS [MH⁺]281 consistent with molecular formula C₁₂H₁₃³⁵ClN₄O₂

### EXAMPLE 1

### N-(3-Bromophenyl)-1-methyl-7-(1-piperidinylcarbonyl)-1H-imidazo[4,5-c]pyridin-4-amine hydrochloride salt

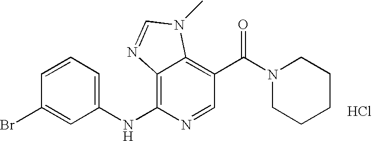

Sodium 4-[(3-bromophenyl)amino]-1-methyl-1H-imidazo[4,5-c]pyridine-7-carboxylate (250 mg including sodium hydroxide) was placed in a boiling tube where it was combined with hydroxybenzotriazole hydrate (107 mg), N-(3-dimethylaminopropyl)-N-ethylcarbodiimide (123 mg), N-ethylmorpholine (0.183 ml), piperidine (0.092 ml) and this was dissolved in dimethylformamide (8 ml). The reaction was stirred at room temperature for 48 hours. The reaction mixture was reduced in vacuo and acidified using 2N hydrochloric acid and then reduced in vacuo. The resulting solid was combined with hydroxybenzotriazole hydrate (107 mg), N-(3-dimethylaminopropyl)-N-ethylcarbodiimide (123 mg), piperidine (0.092 ml), excess N-ethylmorpholine and this was dissolved in dimethylformamide (8 ml). This was then stirred for 24 hours at room temperature. The reaction mixture was reduced in vacuo and combined with water and dichloromethane. The organic layer was collected via a hydrophobic frit and reduced in vacuo. The residue was purified using silica chromotography (10 g cartridge, eluting with 1-2% of 2M ammonia in methanol in dichloromethane). The resultant solution was reduced in vacuo and then purified using mass directed HPLC. The correct fractions were combined and reduced in vacuo to yield a solid which was dissolved in methanol and acetonitrile and 1M hydrochloric acid in diethyl ether added. The solution was reduced in vacuo to yield a solid which was dissolved in 1,4-dioxane and water and placed on a freeze dryer to give a white solid (136 mg).

LC/MS [MH⁺]416 consistent with molecular formula C₁₉H₂₀⁸¹BrN₅O

### EXAMPLE 2

### N-(3-Bromophenyl)-1-methyl-7-(4-morpholinylcarbonyl)-1H-imidazo[4,5-c]pyridin-4-amine hydrochloride salt

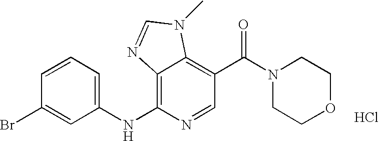

The title compound was prepared in a manner similar to Example 1 from sodium 4-[(3-bromophenyl)amino]-1-methyl-1H-imidazo[4,5-c]pyridine-7-carboxylate (250 mg) where morpholine (94 μl) was used in the coupling procedure. A white solid was obtained (77 mg).

LC/MS [MH⁺]418 consistent with molecular formula C₁₈H₁₈⁸¹BrN₅O₂

### EXAMPLE 3

### N-(3-Bromophenyl)-1-methyl-7-(1-pyrrolidinylcarbonyl)-1H-imidazo[4,5-c]pyridin-4-amine hydrochloride salt

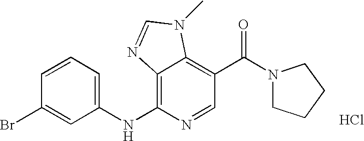

The title compound was prepared in a manner similar to Example 1 from sodium 4-[(3-bromophenyl)amino]-1-methyl-1H-imidazo[4,5-c]pyridine-7-carboxylate (250 mg) where pyrrolidine (89 μl) was used in the coupling procedure. A white solid was obtained (154 mg).

LC/MS [MH⁺]402 consistent with molecular formula C₁₈H₁₈⁸¹BrN₅O

### EXAMPLE 4

### 4-[(3-Bromophenyl)amino]-1-methyl-N-(2-methylpropyl)-1H-imidazo[4,5-c]pyridine-7-carboxamide hydrochloride salt

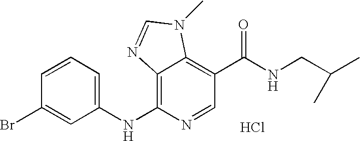

The title compound was prepared in a manner similar to Example 1 from sodium 4-[(3-bromophenyl)amino]-1-methyl-1H-imidazo[4,5-c]pyridine-7-carboxylate (250 mg) where isobutylamine (108 μl) was used in the coupling procedure. Except when the reaction mixture was dried in vacuo and combined with dichloromethane and water, a precipitate remained which was filtered then washed with 30% acetonitrile in water to give a white solid. This was dissolved in methanol and 1M hydrochloric acid in diethyl ether added. The solvent was removed in vacuo to yield a solid which was dissolved in 1,4-dioxane and water and placed on a freeze dryer to give a white solid (154 mg).

LC/MS [MH⁺]404 consistent with molecular formula C₁₈H₂₀⁸¹BrN₅O

### EXAMPLE 5

### N-(2,4-Dichlorophenyl)-1-methyl-7-(4-morpholinylcarbonyl)-1H-imidazo[4,5-c]pyridin-4-amine hydrochloride salt

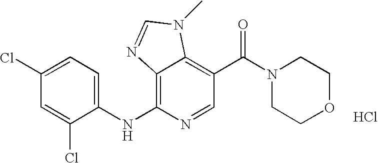

4-[(2,4-Dichlorophenyl)amino[-1-methyl-1H-imidazo[4,5-c]pyridine-7-carboxylic acid hydrochloride salt (135 mg) was placed in a boiling tube where it was combined with hydroxybenzotriazole hydrate (59 mg), N-(3-dimethylaminopropyl)-N-ethylcarbodiimide (68 mg), N-ethylmorpholine (0.1 ml), morpholine (0.052 ml) and this was dissolved in dimethylformamide (8 ml). The reaction was stirred at room temperature for 24 hours. The reaction mixture was then dried in vacuo and combined with water and dichloromethane. The organic layer was collected with a hydrophobic frit, reduced in vacuo and purified on a C-18 cartridge (5 g) eluting from 0-50% acetonitrile in water. The correct fractions were combined and reduced in vacuo, to yield a solid which was dissolved in acetonitrile and 1M hydrochloric acid in diethyl ether added. This was then dried in vacuo to give a solid. The solid was then dissolved in 1,4-dioxane and water and placed on a freeze dryer to give a white solid (44 mg)

LC/MS [MH⁺]406 consistent with molecular formula C₁₈H₁₇³⁵Cl₂N₅O₂

### EXAMPLE 6

### N-(2,4-Dichlorophenyl)-1-methyl-7-(1-piperidinylcarbonyl)-1H-imidazo[4,5-c]pyridin-4-amine hydrochloride salt

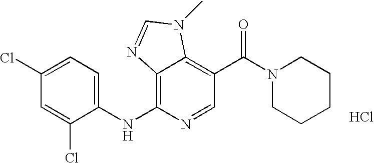

The title compound was prepared in a manner similar to Example 5 from 4-[(2,4-Dichlorophenyl)amino]-1-methyl-1H-imidazo[4,5-c]pyridine-7-carboxylic acid hydrochloride salt (135 mg) where piperdine (51 μl) was used in the coupling procedure. A white solid was obtained (19 mg)

LC/MS [MH⁺]404 consistent with molecular formula C₁₉H₁₉³⁵Cl₂N₅O

### EXAMPLE 7

### N-(2,4-Dichlorophenyl)-1-methyl-7-(1-pyrrolidinylcarbonyl)-1H-imidazo[4,5-c]pyridin-4-amine hydrochloride salt

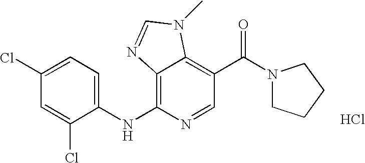

The title compound was prepared a manner similar to Example 5 from 4-[(2,4-Dichlorophenyl)amino]-1-methyl-1H-imidazo[4,5-c]pyridine-7-carboxylic acid hydrochloride salt (135 mg) where pyrrolidine (50 μl) was used in the coupling procedure. A white solid was obtained (37 mg).

LC/MS [MH⁺]390 consistent with molecular formula C₁₈H₁₇³⁵Cl₂N₅O

### EXAMPLE 8

### 4-1(2,4-Dichlorophenyl)amino]-1-methyl-N-(2-methylpropyl)-1H-imidazo[4,5-c]pyridine-7-carboxamide hydrochloride salt

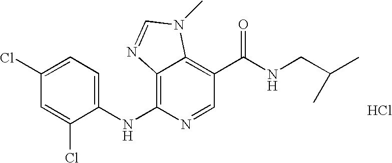

The title compound was prepared a manner similar to Example 5 from 4-[(2,4-Dichlorophenyl)amino]-1-methyl-1H-imidazo[4,5-c]pyridine-7-carboxylic acid hydrochloride salt (135 mg) where isobutylamine (60 μl) was used in the coupling procedure. Except the reaction mixture was reduced in vacuo, the residue partially dissolved in acetonitrile and dimethylsulfoxide. The remaining solid was filtered and dried in vacuo, then dissolved in methanol and 1M hydrochloric acid in diethyl ether added. This was then dried in vacuo to give a solid. The solid was then dissolved in 1,4-dioxane and water and placed on a freeze dryer to give a white solid (42 mg)

LC/MS [MH⁺]392 consistent with molecular formula C₁₈H₁₉³⁵Cl₂N₅O

### EXAMPLE 9a

### N-(3-Chlorophenyl)-1-methyl-7-(4-morpholinylcarbonyl)-1H-imidazo[4,5-c]pyridin-4-amine hydrochloride salt.

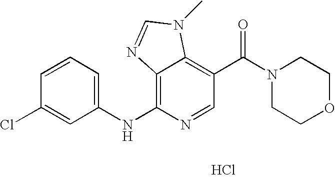

4-[(3-Chlorophenyl)amino]-1-methyl-1H-imidazo[4,5-c]pyridine-7-carboxylic acid (275 mg, 0.91 mmol), dimethylformamide (8 ml), 4-ethylmorpholine (230μl, 1.8 mmol), morpholine (120 μl, 1.36 mmol), 1-hydroxybenzotriazole hydrate (135 mg, 1 mmol) and 1-(3-dimethylaminopropyl)-3-ethylcarbodiimide hydrochloride (155 mg, 1 mmol) were added together and the solution stirred at room temperature overnight. The solvents were evaporated. The residue was partitioned between water and dichloromethane using a hydrophobic frit. The dichloromethane extract was evaporated and purified by chromatography (10 g of silica) eluting with dichloromethane. The column was washed with 3 column volumes of dichloromethane, 2 column volumes of 2% (2M ammonia in methanol)/dichloromethane, 2 column volumes of 5% (2M ammonia in methanol)/dichloromethane, and 2 column volumes of 10% (2M ammonia in methanol)/dichloromethane. The sample was treated with an excess of ethereal hydrogen chloride (5 ml) and then freeze dried to obtain title compound as an off white solid (177 mg).

LC/MS [MH⁺]372 consistent with molecular formula C₁₈H₁₈³⁵ClN₅O₂

### EXAMPLE 9b

### N-(3-Chlorophenyl)-1-methyl-7-(4-morpholinylcarbonyl)-1H-imidazo[4,5-c]pyridin-4-amine

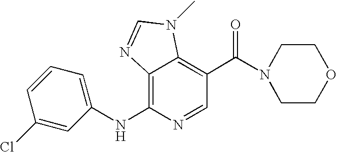

To a stirred suspension of 4-[(3-chlorophenyl)amino]-1-methyl-1H-imidazo[4,5-c]pyridine-7-carboxylic acid (27.19 g, 0.09 moles) in DMF (680 ml) was added N,N-diisopropylethylamine (78.26 ml, 0.45 moles), O-(1H-benzotriazol-1-yl)-N,N,N′,N′-tetramethyluronium hexafluorophosphate (51.18 g 0.135 moles). At this point the reaction starts to become thicker. To this mixture was added morpholine (15.72 ml, 0.18 moles) slowly over 5 minutes. The reaction forms a dark solution. The reaction mixture was stirred at room temperature for 2 hours. The reaction was evaporated to remove 595 ml of DMF. The dark brown oil was taken up into ethyl acetate (3 litres) and this was then successively washed with water (1 litre), aqueous saturated sodium hydrogen carbonate solution (1 litre). A fine precipitate forms in the ethyl acetate layer and this was filtered off. The ethyl acetate layer was washed successively with water (1 litre), 2M aqueous sodium hydroxide (2×500 ml), water (1 litre) and brine (1 litre). The ethyl acetate layer was dried (MgSO₄) and evaporated to give a light brown solid. This was taken up in DCM (200 ml) containing methanol (20 ml), to which was added silica (125 g), and the mixture evaporated. The solid was chromatographed on a Biotage Flash 75 eluting with DCM/methanol (97:3) to give a pale yellow solid which was dried under vacuum at 60 ° C. overnight. The solid obtained was taken up into aqueous 2M hydrochloric acid solution (1 litre), this solution was washed with ethyl acetate (2×500 ml). The aqueous phase was then basified using solid sodium hydrogen bicarbonate to a pH 8. The precipitate formed was filtered off and re-suspended in water(l litre) and stirred for 30 minutes, the solid was filtered off and dried under vacuum at 40° C. overnight to give N-(3-chlorophenyl)-1-methyl-7-(4-morpholinylcarbonyl)-1H-imidazo[4,5-c]pyridin-4-amine (25.01 g 74%) as an off white solid.

NMR (400 MHz, DMSO-d6) HNC122148 δ3.30-3.90 (11H, m), 6.96-6.99 (1H, m), 7.27-7.31 (1H, t), 7.92-7.94 (2H, m), 8.29 (1H, s), 8.33-8.34 (1H, m), 9.51 (1H, s). Consistent with proposed structure

LC/MS, Product retention time 2.23 min, [MH⁺]372 consistent with the molecular formula C₁₈H₁₈³⁵ClN₅O₂

### EXAMPLE 10

### N-(3-Chlorophenyl)-1-methyl-7-(1-piperidinylcarbonyl)-1H-imidazo[4,5-c]pyridin-4-amine hydrochloride salt.

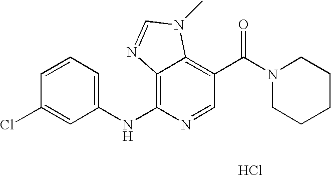

The title compound was prepared in a manner similar to Example 9a from 4-[(3-chlorophenyl)amino]-1-methyl-1H-imidazo[4,5-c]pyridine-7-carboxylic acid (275 mg). Where piperidine (120 μl) was used in the coupling procedure. A white solid was obtained (250 mg).

LC/MS [MH⁺]370 consistent with molecular formula C₁₉H₂₀³⁵ClN₅O

### EXAMPLE 11

### N-(3-Chlorophenyl)-1-methyl-7-(1-pyrrolidinylcarbonyl)-1H-imidazo[4,5-c]pyridin-4-amine hydrochloride salt.

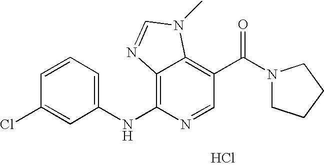

The title compound was prepared in a manner similar to Example 9a from 4-[(3-chlorophenyl)amino]-1-methyl-1H-imidazo[4,5-c]pyridine-7-carboxylic acid (275 mg) where pyrrolidine (110 μl) was used in the coupling procedure. A white solid was obtained (103 mg).

LC/MS [MH⁺]356 consistent with molecular formula C₁₈H₁₈³⁵ClN₅O

### EXAMPLE 12

### 4-[(3-Chlorophenyl)amino]-1-methyl-N-(2-methylpropyl)-1H-imidazo[4,5-c]pyridine-7-carboxamide hydrochloride salt.

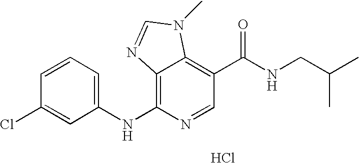

The title compound was prepared a manner similar to Example 9a from 4-[(3-chlorophenyl)amino]-1-methyl-1H-imidazo[4,5-c]pyridine-7-carboxylic acid (275 mg) where isobutylamine (73 μl) was used in the coupling procedure. An off white solid was obtained (144 mg).

LC/MS [MH⁺]358 consistent with molecular formula C₁₈H₂₀³⁵ClN₅O

### EXAMPLE 13

### 4-[(3-Chlorophenyl)oxy]-1-methyl-7-(1-piperidinylcarbonyl)-1H-imidazo[4,5-c]pyridine hydrochloride salt.

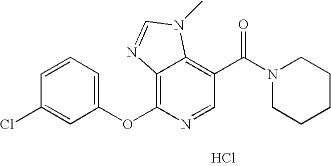

4-[(3-Chlorophenyl)oxy]-1-methyl-1H-imidazo[4,5-c]pyridine-7-carboxylic acid hydrochloride salt (325 mg, 1.07 mmol), dimethylformamide (8 ml), 4-ethylmorpholine (230 μl, 1.8 mmol), piperidine (140 μl, 1.66 mmol), 1-hydroxybenzotriazole hydrate (165 mg, 1.1 mmol) and 1-(3-dimethylaminopropyl)-3-ethylcarbodiimide hydrochloride (190 mg, 1.1 mmol) were added together and the solution stirred at room temperature overnight. The solvents were evaporated. The residue was partitioned between water and dichloromethane using a hydrophobic frit. The dichloromethane extract was evaporated and purified by chromatography (10 g of silica) eluting with dichloromethane. The column was washed with 3 column volumes of dichloromethane, 2 column volumes of 2% (2M ammonia in methanol)/dichloromethane, 2 column volumes of 5% (2M ammonia in methanol)/dichloromethane, and 2 column volumes of 10% (2M ammonia in methanol)/dichloromethane. The sample was treated with hydrogen chloride (1M) solution in diethyl ether (approx 1-2 ml) and then evaporated to dryness. The sample was then dissolved in a combination of 1,4 dioxane and water and freeze dried overnight to obtain title compound as an off white solid (280 mg).

LC/MS [MH⁺]371 consistent with molecular formula C₁₉H₁₉³⁵ClN₄O₂

### EXAMPLE 14

### 4-1(3-Chlorophenyl)oxy]-1-methyl-7-(4-morpholinylcarbonyl)-1H-imidazo[4,5-c]pyridine hydrochloride salt.

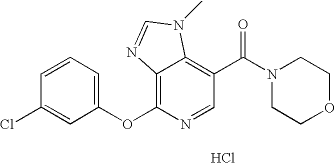

The title compound was prepared in a manner similar to Example 13 from 4-[(3-chlorophenyl)oxy]-1-methyl-1H-imidazo[4,5-c]pyridine-7-carboxylic acid hydrochloride salt (325 mg) where morpholine (140 μl) was used in the coupling procedure. An off white solid was obtained (182 mg).

LC/MS [MH⁺]373 consistent with molecular formula C₁₈H₁₇³⁵ClN₄O₃

### EXAMPLE 15

### 4-1(3-Chlorophenyl)oxy]-1-methyl-7-(1-pyrrolidinylcarbonyl)-1H-imidazo[4,5-c]pyridine hydrochloride salt.

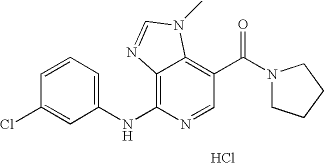

The title compound was prepared in a manner similar to Example 13 from 4-[(3-chlorophenyl)oxy]-1-methyl-1H-imidazo[4,5-c]pyridine-7-carboxylic acid hydrochloride salt (325 mg) where pyrrolidine (120 μl) was used in the coupling procedure. An off white solid was obtained (300 mg).

LC/MS [MH⁺]357 consistent with molecular formula C₁₈H₁₇³⁵ClN₄O₂

### EXAMPLE 16

### 4-1(3-Chlorophenyl)oxy]-1-methyl-N-(2-methylpropyl)-1H-imidazo[4,5-c]pyridine-7-carboxamide hydrochloride salt.

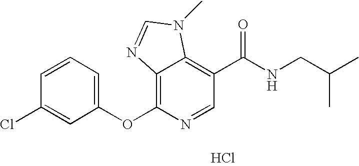

The title compound was prepared in a manner similar to Example 13 from 4-[(3-chlorophenyl)oxy]-1-methyl-1H-imidazo[4,5-c]pyridine-7-carboxylic acid hydrochloride salt (325 mg) where isobutylamine (120 μl) was used in the coupling procedure. An off white solid was obtained (248 mg).

LC/MS [MH⁺]359 consistent with molecular formula C₁₈H₁₉³⁵ClN₄O₂

### EXAMPLE 17

### 1-Methyl-7-(4-morpholinylcarbonyl)-N-{3-[(trifluoromethyl)oxy]phenyl}-1H-imidazo[4,5-c]pyridin-4-amine hydrochloride salt

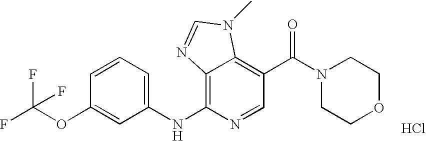

A mixture of 4-chloro-1-methyl-7-(4-morpholinylcarbonyl)-1H-imidazo[4,5-c]pyridine (150 mg), methanesulfonic acid (0.207 ml) and 3-trifluoromethoxyaniline (0.143 ml) in 1,4-dioxane (5 ml) was heated under microwave conditions at 180° C. for thirty minutes. The mixture was concentrated in vacuo, purified by MDAP, suspended in methanol treated with 2N hydrochloric acid in ether (0.5 ml), evaporated and dried to afford the title compound (27 mg).

LC/MS [MH⁺]422 consistent with molecular formula C₁₉H₁₈F₃N₅O₃

### EXAMPLE 18

### N-(3-Fluorophenyl)-1-methyl-7-(4-morpholinylcarbonyl)-1H-imidazo[4,5-c]pyridin-4-amine hydrochloride salt

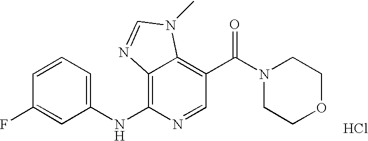

The title compound (36 mg) was prepared in a manner similar to Example 17 from 4-chloro-1-methyl-7-(4-morpholinylcarbonyl)-1H-imidazo[4,5-c]pyridine (150 mg) and 3-fluoroaniline (0.103 ml) except that the reaction time was fifteen minutes.

LC/MS [MH⁺]356 consistent with molecular formula C₁₈H₁₈FN₅O₂

### EXAMPLE 19

### N-(3,4-Difluorophenyl)-1-methyl-7-(4-morpholinylcarbonyl)-1H-imidazo[4,5-c]pyridin-4-amine hydrochloride salt

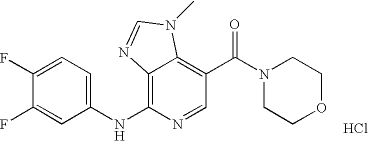

The title compound (72 mg) was prepared in a manner similar to Example 17 from 4-chloro-1-methyl-7-(4-morpholinylcarbonyl)-1H-imidazo[4,5-c]pyridine (150 mg) and 3,4-difluoroaniline (0.106 ml) except that the reaction time was fifteen minutes.

LC/MS [MH⁺]374 consistent with molecular formula C₁₈H₁₇F₂N₅O₂

### EXAMPLE 20

### 1-Methyl-N-[2-methyl-3-(trifluoromethyl)phenyl]-7-(4-morpholinylcarbonyl)-1H-imidazo[4,5-c]pyridin-4-amine hydrochloride salt

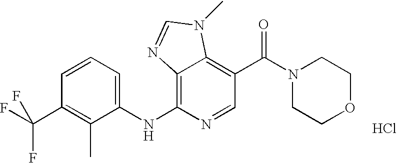

The title compound (32 mg) was prepared in a manner similar to Example 17 from 4-chloro-1-methyl-7-(4-morpholinylcarbonyl)-1H-imidazo[4,5-c]pyridine (150 mg) and 2-methyl-3-trifluoromethylaniline (187 mg) except that the reaction time was fifteen minutes.

LC/MS [MH⁺]420 consistent with molecular formula C₂₀H₂₀F₃N₅O₂

### EXAMPLE 21

### N-[2-Fluoro-3-(trifluoromethyl)phenyl]-1-methyl-7-(4-morpholinylcarbonyl)-1H-imidazo[4,5-c]pyridin-4-amine hydrochloride salt

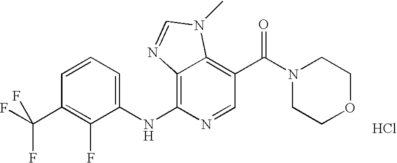

The title compound (33 mg) was prepared in a manner similar to Example 17 from 4-chloro-1-methyl-7-(4-morpholinylcarbonyl)-1H-imidazo[4,5-c]pyridine (150 mg) and 2-fluoro-3-trifluoromethylaniline (0.138 ml) except that the reaction time was twenty minutes. The title compound was an oil and had to be co-evaporated from dichloromethane to afford a foam/solid.

LC/MS [MH⁺]424 consistent with molecular formula C₁₉H₁₇F₄N₅O₂

### EXAMPLE 22

### N-(3-Chloro-4-fluorophenyl)-1-methyl-7-(4-morpholinylcarbonyl)-1H-imidazo[4,5-c]pyridin-4-amine hydrochloride salt

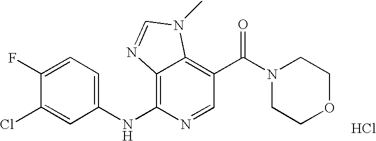

The title compound (57 mg) was prepared in a manner similar to Example 17 from 4-chloro-1-methyl-7-(4-morpholinylcarbonyl)-1H-imidazo[4,5-c]pyridine (150 mg) and 3-chloro-4-fluoroaniline (156 mg) except that the reaction time was twenty minutes. The title compound was further purified by trituration with hexane to afford a white solid.

LC/MS [MH⁺]390 consistent with molecular formula C₁₈H₁₇³⁵Cl FN₅O₂

Formulations for pharmaceutical use incorporating compounds of the present invention can be prepared in various forms and with numerous excipients. Examples of such formulations are given below.

### EXAMPLE 23

### Inhalant Formulation

A compound of formula (I) or a pharmaceutically acceptable derivative thereof, (1 mg to 100 mg) is aerosolized from a metered dose inhaler to deliver the desired amount of drug per use.

### EXAMPLE 24

### Tablet Formulation

| Tablets/Ingredients | | Per Tablet |
|---|---|---|
| 1. | Active ingredient | 40 mg |
| | (Compound of formula (I) or | |
| | pharmaceutically acceptable derivative) | |
| 2. | Corn Starch | 20 mg |
| 3. | Alginic acid | 20 mg |
| 4. | Sodium Alginate | 20 mg |
| 5. | Mg stearate | 1.3 mg |

### Procedure for Tablet Formulation:

Ingredients 1, 2, 3 and 4 are blended in a suitable mixer/blender. Sufficient water is added portion-wise to the blend with careful mixing after each addition until the mass is of a consistency to permit its conversion to wet granules. The wet mass is converted to granules by passing it through an oscillating granulator using a No. 8 mesh (2.38 mm) screen. The wet granules are then dried in an oven at 140° F. (60° C.) until dry. The dry granules are lubricated with ingredient No. 5, and the lubricated granules are compressed on a suitable tablet press.

### EXAMPLE 25

### Parenteral Formulation

A pharmaceutical composition for parenteral administration is prepared by dissolving an appropriate amount of a compound of formula (I) in polyethylene glycol with heating. This solution is then diluted with water for injections Ph Eur. (to 100 ml). The solution is then rendered sterile by filtration through a 0.22 micron membrane filter and sealed in sterile containers.

1. A compound of formula (I): 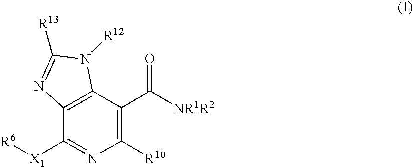
wherein:
X₁is NR⁴ or O;
R¹ is selected from hydrogen, C₁₋₆alkyl, C₃₋₆cycloalkyl and halosubstitutedC₁₋₆ alkyl;
R² is hydrogen or (CH₂)ₘR³ where m is 0 or 1;
or R¹ and R² together with N to which they are attached form an optionally substituted 4- to 8-membered non-aromatic heterocyclyl ring;
R³is a 4- to 8-membered non-aromatic heterocyclyl group, a C₃₋₈ cycloalkyl group, a straight or branched C₁₋₁₀alkyl, a C₂₋₁₀alkenyl, a C₃₋₈cycloalkenyl, a C₂₋₁₀alkynyl, a C₃₋₈cycloalkynyl or phenyl group, any of which can be unsubstituted or substituted, or R⁵;
R⁴is selected from hydrogen, C₁₋₆ alkyl, C₃₋₆ cycloalkyl, halosubstitutedC₁₋₆ alkyl, COCH₃, and SO₂Me;
R⁵ is 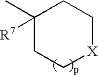
wherein p is 0, 1 or 2, and X is CH₂, O, S, or SO₂;
R⁶ is unsubstituted or substituted phenyl, unsubstituted or substituted C₃₋₆cycloalkyl or an unsubstituted or substituted 4- to 8-membered non-aromatic heterocyclyl ring;
R⁷ is OH, C₁₋₆alkoxy, NR^{8a}R^{8b}, NHCOR⁹, NHSO₂R⁹ or SO_{q}R⁹;
R^{8a} is H or C₁₋₆alkyl;
R^{8b} is H or C₁₋₆alkyl;
R⁹ is C₁₋₆alkyl;
R¹⁰ is hydrogen, substituted or unsubstituted (C₁₋₆)alkyl or chloro;
R¹² is hydrogen or C₁₋₆alkyl;
R¹³ is hydrogen or C₁₋₆alkyl;
q is 0, 1 or 2;
or a pharmaceutically acceptable derivative thereof.

2. The compound as claimed in claim 1 wherein R¹ is hydrogen.

3. The compound as claimed in claim 1 wherein R² is (CH₂)ₘR³ where m is 0 or 1.

4. The compound as claimed in claim 1 wherein R³ is an unsubstituted or substituted C₁₋₆ alkyl group.

5. The compound as claimed in claim 1 wherein R¹ and R² together with the nitrogen to which they are attached form a morpholinyl, pyrrolidinyl or piperidinyl ring.

6. The compound as claimed in claim 1 wherein R⁶ is an unsubstituted or substituted phenyl group.

7. The compound as claimed in claim 1 wherein X₁ is NR⁴.

8. The compound as claimed in claim 1 wherein R⁴ is C₁₋₆ alkyl or hydrogen.

9. The compound as claimed in claim 1 wherein R¹⁰ is hydrogen.

10. The compound as claimed in claim 1 wherein R¹² is methyl.

11. The compound as claimed in claim 1 wherein R¹³ is hydrogen.

12. A compound of formula (Ia): 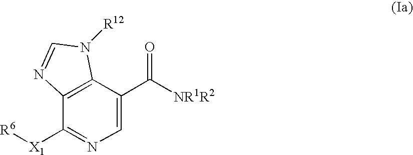
wherein
X₁ is NR⁴;
R¹ is hydrogen;
R² is (CH₂)ₘR³ where m is 0 or 1;
or R¹ and R² together with N to which they are attached form a morpholinyl, pyrrolidinyl, or piperidinyl ring of which may be unsubstituted or substituted;
R³ is an unsubstituted or substituted straight or branched C₁₋₆alkyl;
R⁴ is hydrogen or methyl,
R⁶ is unsubstituted or substituted phenyl;
R¹² is hydrogen or methyl;
or a pharmaceutically acceptable derivative thereof.

13. A pharmaceutical composition comprising a compound as claimed in claim 1 or a pharmaceutically acceptable derivative thereof and a pharmaceutical carrier or diluent thereof.

14. (canceled)

15. The pharmaceutical composition as claimed in claim 13 further comprising a second therapeutic agent.

16. (canceled)

17. (canceled)

18. (canceled)

19. A method of treating mammal suffering from a condition which is mediated by the activity of cannabinoid 2 receptor which comprises administering to said subject a non toxic, therapeutically effective amount of a compound of formula (I) as claimed in claim 1 or a pharmaceutically acceptable derivative thereof.

20. The method of treatment as claimed in claim 19 wherein the condition which is mediated by the activity of cannabinoid 2 receptor is an immune disorder, an inflammatory disorder, pain, rheumatoid arthritis, multiple sclerosis, osteoarthritis or osteoporosis.

21. The method as claimed in claim 20 wherein the pain is selected from inflammatory pain, viseral pain, cancer pain, neuropathic pain, lower back pain, muscular sceletal, post operative pain, acute pain and migraine.
